SEQUENCE LISTING (36 sequences)

SEQ ID NO: 1 - DNA, 23 bp, artificial sequence
  sense strand of HO-1 siRNA sequence 1
aaggacauggccuucugguautt

SEQ ID NO: 2 - DNA, 23 bp, artificial sequence
  antisense strand of HO-1 siRNA sequence 1
auaccagaaggccauguccuutt

SEQ ID NO: 3 - DNA, 23 bp, artificial sequence
  sense strand of HO-1 siRNA sequence 2
aaugaacacucuggagaugactt

SEQ ID NO: 4 - DNA, 23 bp, artificial sequence
  antisense strand of HO-1 siRNA sequence 2
gucaucuccagaguguucauutt

SEQ ID NO: 5 - DNA, 23 bp, artificial sequence
  sense strand of HO-1 siRNA sequence 3
aagaccagagucccucacagatt

SEQ ID NO: 6 - DNA, 23 bp, artificial sequence
  antisense strand of HO-1 siRNA sequence 3
ucugugagggacucuggucuutt

SEQ ID NO: 7 - DNA, 23 bp, artificial sequence
  sense strand of HO-1 siRNA sequence 4
aagccacacagcacuauguaatt

SEQ ID NO: 8 - DNA, 23 bp, artificial sequence
  antisense strand of HO-1 siRNA sequence 4
uuacauagugcuguguggcuutt

SEQ ID NO: 9 - DNA, 23 bp, artificial sequence
  sense strand of HO-1 siRNA sequence 5
aagccgagaaugcugaguucatt

SEQ ID NO: 10 - DNA, 23 bp, artificial sequence
  antisense strand of HO-1 siRNA sequence 5
ugaacucagcauucucggcuutt

SEQ ID NO: 11 - DNA, 21 bp, artificial sequence
  sense strand of HO-1 siRNA sequence 6
ggagauugagcgcaacaagtt

SEQ ID NO: 12 - DNA, 21 bp, artificial sequence
  antisense strand of HO-1 siRNA sequence 6
cuuguugcgcuaaaucucctt

SEQ ID NO: 13 - DNA, 21 bp, artificial sequence
  sense strand of HO-1 siRNA sequence 7
ugauagaagaggccaagactt

SEQ ID NO: 14 - DNA, 21 bp, artificial sequence
  antisense strand of HO-1 siRNA sequence 7
gucuuggccucuucuaucatt

SEQ ID NO: 15 - DNA, 21 bp, artificial sequence
  sense strand of HO-1 siRNA sequence 8
cugcguuccugcucaacautt

SEQ ID NO: 16 - DNA, 21 bp, artificial sequence
  antisense strand of HO-1 siRNA sequence 8
auguugagcaggaacgcagtt

SEQ ID NO: 17 - DNA, 21 bp, artificial sequence
  sense strand of nonspecific siRNA sequence 9
gcgcgcuuuguaggauucgtt

SEQ ID NO: 18 - DNA, 21 bp, artificial sequence
  antisense strand of nonspecific siRNA sequence 9
cgaauccuacaaagcgcgctt

SEQ ID NO: 19 - DNA, 21 bp, artificial sequence
  sense strand of nonspecific siRNA sequence 10
uucuccgaacgugucacgutt

SEQ ID NO: 20 - DNA, 21 bp, artificial sequence
  antisense strand of nonspecific siRNA sequence 10
acgugacacguucggagaatt

SEQ ID NO: 21 - DNA, 21 bp, artificial sequence
  sense strand of nonspecific siRNA sequence 11
gcgcgcuuuguaggauucgtt

SEQ ID NO: 22 - DNA, 21 bp, artificial sequence
  antisense strand of nonspecific siRNA sequence 11
cgaauccuacaaagcgcgctt

SEQ ID NO: 23 - DNA, 21 bp, artificial sequence
  primer
tcccagacaccgctcctccag

SEQ ID NO: 24 - DNA, 21 bp, artificial sequence
  primer
ggatttggggctgctggtttc

SEQ ID NO: 25 - DNA, 21 bp, artificial sequence
  primer
gtgggccgctctaggcaccaa

SEQ ID NO: 26 - DNA, 24 bp, artificial sequence
  primer
ctctttgatgtcacgcacgatttc

SEQ ID NO: 27 - RNA, 21 nt, artificial sequence
  target sequence of mouse STAT3 siRNA
aacaaccuucgaagaaucaag

SEQ ID NO: 28 - DNA, 23 bp, artificial sequence
  sense strand of STAT3 siRNA sequence 12
caaccuucgaagaaucaaguutt

SEQ ID NO: 29 - DNA, 23 bp, artificial sequence
  antisense strand of STAT3 siRNA sequence 12
cuugauucuucgaagguuguutt

SEQ ID NO: 30 - DNA, 107 bp, artificial sequence
  RT-PCR product
acctgaaccggcatctgcacacctggatccaggataacggaggctgggatgcctttgtggaactatatggccccagcatgcgacctctgtttgatttctcctggctg

SEQ ID NO: 31 - DNA, 21 bp, artificial sequence
  primer
acctgaaccggcatctgcaca

SEQ ID NO: 32 - DNA, 24 bp, artificial sequence
  primer
cagccaggagaaatcaaacagagg

SEQ ID NO: 33 - DNA, 23 bp, artificial sequence
  sense strand of VEGF siRNA sequence 13
augugaaugcagaccaaagaatt

SEQ ID NO: 34 - DNA, 23 bp, artificial sequence
  antisense strand of VEGF siRNA sequence 13
uucuuuggucugcauucacautt

SEQ ID NO: 35 - DNA, 20 bp, artificial sequence
  primer
cctccgcggccatgaacttt

SEQ ID NO: 36 - DNA, 22 bp, artificial sequence
  primer
tctttccggatccgagatctgg

(19) United States
(12) Patent Application Publication
(10) Pub. No.: US 2005/0265927 A1
(43) Pub. Date: Dec. 1, 2005

(54) Intranasal delivery of nucleic acid molecules

(75) Inventors: Patty Lee, Guilford, CT (US); Xuchen Zhang, Orange, CT (US)

Correspondence Address: PATREA L. PABST;PABST PATENT GROUP LLP, 400 COLONY SQUARE, SUITE 1200, ATLANTA, GA, 30361, US

(73) Assignee: Yale University

(21) Appl. No.: 11/120,370

(22) Filed: May 3, 2005

Related U.S. Application Data
(60) Provisional application No. 60/571,796, filed on May 17, 2004.

(51) Int. Cl. A61K 48/00; A61L 9/04; A61K 9/14

(52) U.S. Cl. 424/46; 514/44

(57) ABSTRACT

Aerosol delivery of nucleic acids to the lungs using viral vectors, polymers, surfactants, or excipients has been described. Compositions for intranasal administration are described that contain nucleic acids without viral or plasmid vectors and with little to no polymers, surfactants, or excipients. In one embodiment, the composition for intranasal delivery consists essentially of at least one nucleic acid and an aqueous solution. Suitable nucleic acids for intranasal delivery include, but are not limited to, dsDNA, dsRNA, ssDNA, ssRNA, short interfering RNA, micro-RNA, and antisense RNA. Methods for treatment, diagnosis, or prevention of at least one symptom or manifestation of a lung disease are also described consisting of administration by intranasal delivery an effective amount of a composition containing a nucleic acid. The composition may be formulated as a liquid or aerosol or other acceptable formulation for intranasal administration.

### CROSS-REFERENCE TO RELATED APPLICATIONS

This application claims priority to U.S. Ser. No. 60/571,796 entitled ”Heme Oxygenase-1 (HO-1) siRNA,” filed in the U.S. Patent and Trademark Office on May 17, 2004.

### STATEMENT REGARDING FEDERALLY FUNDED RESEARCH

The Federal Government has certain rights in the invention disclosed herein by virtue of Grant No. HL004034 from the National Institute of Health to Patty Lee.

### FIELD OF THE INVENTION

The present invention relates to compositions and methods of intranasal delivery of nucleic acid molecules.

### BACKGROUND OF THE INVENTION

Lung diseases comprise a spectrum of manifestations and etiologies, and may be particularly difficult to treat with systemic administration of potential therapeutics. Over 150 diseases of the interstitium, the tissue between alveoli, have been identified, including many types of fibrosis. Other lung diseases include disorders of gas exchange, disorders of blood circulation, disorders of the airways, and disorders of the pleura. Lung cancers include both primary lung cancers and metastases from primary cancers of various other organs or tissues. Infectious diseases of the lung include viral, bacterial, and fungal infectious agents. A number of general methods have been described for delivering medically important molecules, including small molecules, nucleic acids, and/or protein or peptide compositions, in an effort to improve bioavailability and/or to target delivery to particular locations within the body. Such methods include the use of prodrugs, encapsulation into liposomes or other particles, and co-administration in uptake enhancing formulations (for review see, e.g., Critical Reviews in Therapeutic Drug Carrier Systems, Stephen D. Bruck, ed., CRC Press, 1991).

Pulmonary administration of therapeutic compositions comprised of low molecular weight drugs, for example, beta-androgenic antagonists to treat asthma, has been observed. Other therapeutic agents that are active in the lungs have been administered systemically and targeted via pulmonary absorption. Nasal delivery is considered to be a promising technique for administration of therapeutics for the following reasons: the nose has a large surface area available for drug absorption due to the coverage of the epithelial surface by numerous microvilli, the subepithelial layer is highly vascularized, the venous blood from the nose passes directly into the systemic circulation and therefore avoids the loss of drug by first-pass metabolism in the liver, it offers lower doses, more rapid attainment of therapeutic blood levels, quicker onset of pharmacological activity, fewer side effects, high total blood flow per cm³, porous endothelial basement membrane, and it is easily accessible.

Gene therapy is generally understood to refer to techniques designed to deliver nucleic acids, including antisense DNA and RNA, ribozymes, viral genome fragments and functionally active therapeutic genes into targeted cells (Culver, 1994. Gene Therapy: A Handbook for Physicians. Mary Ann Liebert, Inc., New York, N.Y.). Such nucleic acids can themselves be therapeutic, as for example antisense DNAs that inhibit mRNA translation, or they can encode, for example, therapeutic proteins that promote, inhibit, augment, or replace cellular functions. Gene therapy holds great promise as a clinical treatment for a variety of human maladies, including lung diseases. The use of common systemic delivery methods, such as intravenous injection, is unattractive because it is typically desired to limit introduction of the transgene to a specific tissue. Furthermore, the transgene may produce a toxic substance. Thus, it is advantageous to limit exposure to the transgene to the tissue of interest.

Virus vectors are among the most efficient gene therapy vectors which have been demonstrated. However, virus vectors sometimes elicit an immune response in the gene therapy host, which can inhibit the therapeutic benefit provided by the vector. Furthermore, use of retrovirus vectors can result in integration of the nucleic acid of the vector into the genome of the host, potentially causing harmful mutations.

Nonviral vectors and methods employing the use of polymers, surfactants, and/or excipients have been employed to introduce nucleic acids into cells including conjugation with a targeting ligand, conjugation with a penetration peptide, derivatization with a lipid and incorporation into liposomes, and cationic liposomes. The majority of non-viral vectors consist of plasmid DNA complexed with lipids or polycations. Many different lipids with ability to deliver plasmid DNA to cells in vitro and in vivo have been reported (Gao, et al., Gene Therapy 2:710-722 (1995)). However, the method of formulation of the lipid-plasmid DNA complexes, particularly the plasmid DNA to lipid ratio, is critical, since it will contribute to particle size and surface charge of the complexes. Lipid-plasmid DNA complexes are most often tested in vitro for convenience. Injection and nebulization of plasmid DNA complexed with water has been described (Gorman, et al., Gene Therapy 4:983-992 (1997) and Crook, et al., Gene Therapy 3:834-839 (1996)). However, it has been demonstrated that when injected into the cytoplasm of cells, plasmid DNA of 1000 base pairs or more diffused slowly from the cytoplasm into the nucleus, preventing efficient gene expression (Lukacs, et al., J Biol Chem. January 21;275(3):1625-9 (2000)). Therefore, the size of plasmid DNA constructs limits their efficacy as therapeutic agents.

The effectiveness of gene therapy depends on efficient delivery of nucleic acids to target cells of interest. Aerosol delivery is an attractive approach because it is non-invasive and has the potential for delivering high concentrations of the therapeutic nucleic acid. Aerosol delivery of nucleic acids to the lungs using viral vectors, polymers, surfactants, or excipients has been described. McDonald, et al., describes aerosol delivery of an adenoviral vector encoding the cystic firbosis transmembrane conductance regulator protein (CFTR) to non-human primates (McDonald, et al., Human Gene Therapy 8:411-422 (1997)). Canonico, et al., describes the in vivo gene transfer of a plasmid containing recombinant human alpha 1-antitrpsin gene and a cytomegalovirus promoter complexed to cationic liposomes to the lungs by aerosol to rabbits (Canonico, et al., Am. J. Respir. Cell Mol. Biol., 10:24-29 (1994)). Stribling, et al., describes that the aerosol delivery of a chloramphenicol acetyltransferase reporter gene complexed to a cationic liposome carrier can produce CAT gene expression in mouse lungs (Stribling, et al., Proc. Natl Acad. Sci. USA 89:11277-11281 (1992)). Massaro, et al., describes delivery of small inhibitory RNA molecules complexed to the lipoprotein pulmonary surfactant, known as surface active material or SAM, to the pulmonary alveoli in mice via liquid deposition into the nasal orifice (Massaro, et al., Am. J. Physiol. Lung Cell Mol. Physiol. 287:L 1066-L1070 (2004)). U.S. patent application No. 2005/0008617 by Chen, et al., describes delivery of RNAi-inducing agents including short-interfering RNA (siRNA), short hairpin RNA (shRNA), and RNAi-inducing vectors complexed with cationic polymers, modified cationic polymers, lipids, and/or surfactants suitable for introduction into the lung. U.S. patent application No. 2003/0157030 by Davis, et al., describes administration of RNAi constructs such as siRNAs or nucleic acids that produce siRNAs complexed with polymers for nasal delivery.

Among the limited number of other reports on siRNA administration in vivo, all use systemic delivery, transfection chemicals, or viral vectors (Hasuwa, et al., FEBS Lett., 532:227-230 (2002); McCaffrey, et al., Nature, 418:38-39 (2002); Reich, et al., Mol. Vis., 9:210-216 (2003); Sorensen, et al., J. Mol. Biol., 327:761-766 (2003); and Zender, et al., Proc. Natl. Acad. Sci U.S.A., 100:7797-7802 (2003)). Pulmonary gene therapy offers the hope of treatment for conditions such as cystic fibrosis, lung cancer, pulmonary fibrosis and acute respiratory distress syndrome for which current therapy is inadequate. Although initial clinical trials in cystic fibrosis and non-small cell lung cancer have shown promise the results have not been as good as might have been anticipated (Jenkins Monaldi Arch Chest Dis. 59(1):17-24 (2003)). The toxicity associated with the use of systemic delivery of nucleic acids and/or a transfection chemical or viral vector raises concerns for clinical use. In addition, the use of plasmid DNA constructs limits efficacy and delivery of plasmid DNA as a potential therapeutic agent.

It is therefore an object of the present invention to provide compositions for intranasal delivery of nucleic acids for the treatment of a number of pulmonary diseases.

It is further an object of the present invention to provide compositions for intranasal administration containing nucleic acids without viral vectors or plasmid vectors and with little to no polymers, surfactants, or excipients.

### BRIEF SUMMARY OF THE INVENTION

Compositions for intranasal administration are described herein that contain nucleic acids without viral or plasmid vectors and with little to no polymers, surfactants, or excipients. In one embodiment, the composition for intranasal delivery consists essentially of at least one nucleic acid molecules and an aqueous solution. Suitable nucleic acids for intranasal delivery include, but are not limited to, dsDNA, dsRNA, ssDNA, ssRNA, short interfering RNA, micro-RNA, and antisense RNA. In one embodiment, the size range of the nucleic acids is 30 nucleotides or less in length, although oligonucleotide molecules of between 5 and up to 60 nucleotides can be utilized. In a preferred embodiment the size range of the nucleic acids is between 19 to 23 nucleotides in length.

The compositions are administered to a patient in need of treatment, prophylaxis or diagnosis of at least one symptom or manifestation (since disease can occur/progress in the absence of symptoms) of a lung disease. In one embodiment, the compositions for intranasal administration are administered in an effective amount to inhibit gene expression, preferably in the lung. The composition is administered in a dose range of 3 to 400 micrograms (μg) per 20 grams (g) of body weight, with upper dosing limit of 1 gram per 20 grams body weight. In a preferred embodiment the composition is administered in a dose range of 50 to 100 μg per 20 g of body weight. In a preferred embodiment the composition is administered in a dose of 150 nanomoles (nM) per kilogram (kg) of body weight. In one embodiment, the composition is administered to inhibit expression of heme-oxygenase-1 (HO-1) in the lung. In another embodiment, the composition is administered in an effective amount to inhibit expression of signal transducer and activator of transcription (STAT3) in the lung.

Methods for treatment, diagnosis, or prevention of at least one symptom or manifestation of a lung disease are also described consisting of administration by intranasal delivery an effective amount of a composition containing a nucleic acid molecule to alleviate at least one symptom or decrease at least one manifestation. The composition may be formulated as a liquid or aerosol or other acceptable formulation for intranasal administration. In one embodiment, the composition delivered by intranasal administration results in inhibition of gene expression in the lung. In another embodiment, the lung specific delivery of the composition is delivered in an effective amount to treat, diagnose, or prevent at least one symptom or manifestation of a lung disease. Suitable lung diseases for treatment diagnosis, or prevention of at least one symptom of the lung disease include but are not limited to, lung cancers; lung inflammatory conditions such as asthma, cystic fibrosis, emphysema, bronchitis, and bronchiectasis; interstitial lung disease and interstitial fibrosis; pneumonia caused by bacterial, viral, fungal, parasitic, and mycobacteria infection; occupational lung diseases such as coal, silica, asbestos, and isocyanates; lung disease secondary to collagen vascular diseases such as systemic lupus erythematosis; rheumatoid arthritis; scleroderma; dermatomyositis; mixed connective tissue disorder; vasculitis associated lung disease such as Wegener granulomatosis and Good-pasture's Syndrome; sarcoid; and the syndrome of Acute Lung Injury/Acute Respiratory Distress Syndrome. In one embodiment where systemic delivery is required, the nucleic acid molecule inhibits expression of heme-oxygenase-1 (HO-1) for treatment of congenital hyperbilirubinemia, Crigler-Najjar syndrome, and hyperbilirubinemia of newborns.

### BRIEF DESCRIPTION OF THE DRAWINGS

FIG. 1A shows HO-1 siRNA significantly increased apoptosis in PAEC during A-R. PAEC were untransfected (control), transfected with nonspecific siRNA (nonspecific siRNA), transfected with HO-1 siRNA directed against rodent siRNA (siRNA), stably transfected with empty retroviral vector (LXSN), stably transfected with human HO-1 overexpressing retroviral vector (LSN/HO-1), HO-1 overexpressors transfected with HO-1 siRNA directed against human HO-1 (LSN/HO-1/Human siRNA), or HO-1 overexpressors transfected with HO-1 siRNAs directed against both human and rodent HO-1 (LSN/HO-1/Double siRNA), and then the cells were exposed to room air (RA), 24-h anoxia (24A) or 24-h anoxia followed by 1-h reoxygenation (A-R). Apoptosis was quantitated by FACS analysis and is represented graphically. Data represent the mean of three independent experiments ±S.E.*, p<0.05, compared with corresponding siRNA 24-h anoxia and A-R; #, p<0.05, compared with corresponding LSN/HO-1/Human siRNA 24-h anoxia and A-R.

FIG. 1B shows HO-1 siRNA significantly increased apoptosis in mouse lung during I-R. Mice were untreated (naive, A) or subjected to lung I-R after no treatment (I-R, B), intranasal nonspecific siRNA at 2 mg/kg of body weight (nonspecific siRNA/I-R, C), intranasal HO-1 siRNA directed against rodent HO-1 (HO-1 siRNA/I-R, D), intranasal empty adenoviral vector at 5×10⁸ plaque-forming units (Ad-X-LacZ/I-R, E), or intranasal adenoviral-HO-1 (Ad-HO-1/I-R, F). Lung sections were then processed for TUNEL staining (arrows indicate representative TUNEL-positive cells). C, graphical quantitation of TUNEL-posi-tive cells in mouse lungs from the conditions shown in B. Data represent the mean of three independent experiments ±S.E.*, p<0.05, compared with naive; #, p<0.05, compared with I-R.

FIGS. 2A, 2B, and 2C show HO-1 siRNA increased Fas expression and caspase 3 activity in PAEC during A-R. PAEC were untransfected (control), transfected with nonspecific siRNA (nonspecific siRNA), transfected with HO-1 siRNA directed against rodent siRNA (siRNA), stably transfected with empty retroviral vector (LXSN), stably transfected with human HO-1-overexpressing retroviral vector (LSN/HO-1), HO-1 overexpressors transfected with HO-1 siRNA directed against human HO-1 (LSN/HO-1/Human siRNA), or HO-1 overexpressors transfected with HO-1 siRNAs directed against both human and rodent HO-1 (LSN/HO-1/Double siRNA), and then the cells were exposed to room air (RA), 24-h anoxia (24A) or 24-h anoxia followed by 1-h reoxygenation (A-R). FIG. 2A, PAEC were stained with anti-Fas or anti-rat IgG (Negative Control) antibody during A-R, and Fas expression was detected by FACS analysis. The data are representative of three independent experiments. FIG. 2B shows graphical quantitation of the percentage of total cells that express Fas during the various conditions. The data represent the mean of three independent experiments ±S.E.*, p<0.05, compared with corresponding siRNA 24-h anoxia and A-R; #, p<0.05, compared with corresponding LSN/HO-1/Human siRNA 24-h anoxia and A-R. FIG. 3C shows caspase 3 activity in PAEC during A-R and is represented graphically. Data represent the mean of three independent experiments ±S.E.*, p<0.05, compared with corresponding siRNA 24-h anoxia and A-R; #, p<0.05, compared with corresponding LSN/HO-1/Human siRNA 24-h anoxia and A-R.

FIG. 3A shows intranasal administration of STAT3 siRNA increases I-R induced expression of intercellular adhesion molecule 1 (ICAM-1), a pro-inflammatory gene, in the lung.

FIG. 3B shows intranasal administration of STAT3 siRNA increases I-R induced expression of vascular cell adhesion molecule 1 (VCAM-1), a pro-inflammatory gene, in the lung.

FIG. 3C shows intranasal administration of STAT3 siRNA increases I-R induced expression of interleukin 1β (IL-1β), a pro-inflammatory gene, in the lung.

FIG. 3D shows intranasal administration of STAT3 siRNA increases I-R induced expression of interleukin 6 (IL-6), a pro-inflammatory gene, in the lung.

FIG. 3E shows intranasal administration of STAT3 siRNA increases I-R induced expression of Fas, a pro-apoptotic gene, in the lung.

FIG. 4 is a graphical representation of percent TUNEL-positive or apoptotic cells following intranasal administration of STAT3 siRNA in mouse lungs during I-R induced lung injury.

FIG. 5A shows intranasal administration of STAT3 siRNA increases I-R induced expression of caspase 3 in the lung.

FIG. 5B shows intranasal administration of STAT3 siRNA increases I-R induced expression of caspase 8 in the lung.

FIG. 6 is a graphical representation of caspase 3 activation represented as fold induction in mouse lungs during I-R induced lung injury.

FIG. 7 is a graphical representation of Malondialdehyde (MDA) products in lung lysates and shows that intranasal administration of STAT3 siRNA increases I-R induced lung oxidative stress during I-R injury in the lung.

FIG. 8 is a graphical representation of the percent TUNEL-positive or apoptotic cells following intranasal administration of Bcl-2 siRNA in mouse lungs during hyperoxia exposure.

FIG. 9 is a graphical representation of LDH activity following transfection of STAT3 siRNA into endothelial cells and hyperoxia exposure.

FIGS. 10A and 10B is a graphical representation of survival as measured in days of mice during hyperoxia exposure following intranasal administration of Bcl-2 siRNA and a Bcl-2 siRNA mismatch control construct.

FIGS. 11A and 11B shows intra nasal Bcl-2 siRNA has no effect on hyperoxia-induced lung injury as assessed by lung cell counts (FIG. 11A) or lung protein levels (FIG. 11B).

FIG. 12 is a graphical representation of percent TUNEL-positive or apoptotic cells following intranasal administration of Bcl-2 siRNA in mouse lungs during I-R induced lung injury.

### DETAILED DESCRIPTION OF THE INVENTION

### DEFINITIONS

As generally used herein a “nucleotide” is a nucleic acid that contains a sugar, deoxyribose or ribose, a base, and a phosphate group. Nucleotides are the monomeric units of nucleic acid polymers. Nucleotides are linked together through the phosphate groups to form nucleic acid.

As generally used herein “nucleic acid” or “nucleic acid molecule” refers to a polymer containing at least two nucleotides. The term nucleic acid includes deoxyribonucleic acid (“DNA”) and ribonucleic acid (“RNA”) and encompasses sequences that include any of the known base analogs of DNA and RNA. Nucleic acids can be linear, circular, or have higher orders of topology (e.g., supercoiled plasmid DNA). DNA can be in the form of antisense, plasmid DNA, parts of a plasmid DNA, vectors (P1, PAC, BAC, YAC, artificial chromosomes), expression cassettes, chimeric sequences, chromosomal DNA, or derivatives of these groups. RNA can be in the form of oligonucleotide RNA, tRNA (transfer RNA), snRNA (small nuclear RNA), rRNA (ribosomal RNA), mRNA (messenger RNA), antisense RNA, (interfering) double-stranded and single-stranded RNA, ribozymes, chimeric sequences, or derivatives of these groups. Nucleic acid can be single (“ssDNA”), double (“dsDNA”), triple (“tsDNA”), or quadruple (“qsDNA”) stranded DNA. RNA can be single stranded RNA (“ssRNA”) or double stranded RNA (“dsRNA”).

As generally used herein, the term “gene” generally refers to a nucleic acid sequence that comprises coding sequences necessary for the production of a therapeutic nucleic acid (e.g., ribozyme) or a polypeptide or precursor. The polypeptide can be encoded by a full-length coding sequence or by any portion of the coding sequence so long as the desired activity or functional properties (e.g., enzymatic activity, ligand binding, signal transduction) of the full-length polypeptide or fragment are retained. The term also encompasses the coding region of a gene and the including sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as “5′ untranslated sequences.” The sequences that are located 3′ or downstream of the coding region and which are present on the mRNA are referred to as “3′ untranslated sequences.” The term gene encompasses both cDNA and genomic forms of a gene. A genomic form or clone of a gene contains the coding region interrupted with “non-coding sequences” termed “introns” or “intervening regions” or “intervening sequences.”

As generally used herein, “introns” are segments of a gene, which are transcribed into nuclear RNA. Introns can contain regulatory elements such as enhancers. Introns are removed or “spliced out” from the nuclear or primary transcript. Introns are absent in the messenger RNA (mRNA) transcript. The mRNA functions during translation to specify the sequence or order of amino acids in a nascent polypeptide. The term non-coding sequences also refers to other regions of a genomic form of a gene including, but not limited to, promoters, enhancers, transcription factor binding sites, polyadenylation signals, internal ribosome entry sites, silencers, insulating sequences, matrix attachment regions. These sequences may be present close to the coding region of the gene (within 10,000 nucleotide) or at distant sites (more than 10,000 nucleotides). These non-coding sequences may influence the level or rate of transcription and translation of the gene.

As generally used herein, the term “gene expression” refers to the process of converting genetic information encoded in a gene into RNA (e.g., mRNA, rRNA, tRNA, or snRNA) through “transcription” of a deoxyribonucleic gene (e.g., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

As generally used herein “antisense” is a nucleic acid that interferes with the function of DNA and/or RNA and can be used suppress expression of a gene.

As generally used herein “interfering RNA” (“RNAi”) is double stranded short-interfering RNA (siRNA) or single-stranded micro-RNA (miRNA) that results in catalytic degradation of specific mRNAs, and can also be used to lower or inhibit gene expression.

I. Compositions

a. Nucleic Acids

Suitable nucleic acids for intranasal delivery include, but are not limited to, dsDNA, dsRNA, ssDNA, ssRNA, short interfering RNA, micro-RNA, and antisense RNA. Such nucleic acids can be therapeutic in that they prevent or treat one or more symptoms of a disease, or they can be diagnostic. Suitable nucleic acids for intranasal delivery can be constructed through a variety of methods known to one of ordinary skill in the art. Nucleic acids suitable for intranasal delivery as described in the present application may also be purchased from a variety of commercial sources. The size range of the nucleic acid molecules is preferably 30 nucleotides of less in length, although it may be possible for the molecules to be up to 60 nucleotides in length. Lengths greater than 30 mer for the double stranded siRNA have been found to elicit an immune response by investigators. More preferably the size range of the nucleic acid molecules is between 19 and 23 nucleotides in length.

Any DNA that would be useful to achieve a desired effect could be used as described herein. Preferably, the DNA is double-stranded or single stranded. More preferably, the DNA is single-stranded. For example, inactivation of gene expression by nucleic acid might also be exerted by triple helix formation between genomic double-stranded DNA and an oligonucleotide. These oligonucleotides can bind with high specificity of recognition to the major groove of double helical DNA by forming Hoogsteen type bonds between the purine bases of the Watson-Crick base pairs, (i.e. between the thymidine and TA base pairs and between protonated cytosine and CG base pairs). A second motif for triple helix recognition of double-stranded DNA is comprised by a homopurine motif in which a purine-rich oligonucleotide binds to DNA antiparallel to the Watson-Crick purine strand. Pyrimidine unmodified oligodeoxynucleotides or backbone-modified oligonucleotides are able to block gene transcription in a sequence specific manner. Oligonucleotides that can bind specifically to double-helical DNA to form a local triple helix structure have been characterized for more than a decade and a wealth of information on the parameters that govern their structure and stability is available (Sun, et al. Curr. Opin. Struct. Biol. 6:327-333 (1996)). Triplex formation within promoter sites has been shown to block transcription factor access and inhibit gene activation in vitro, and several studies have demonstrated that triple helix forming oligonucleotides (TFOs) can decrease gene expression in mammalian cells in a directed way (reviewed in Seidman, et al., J Clin Invest. 112(4):487-94 (2003)). TFOs can also be used to mediate genome modification, resulting in a change in target sequence. This has the advantage of introducing permanent changes in the target sequence. It also has potential as a gene knockout tool and as a means for gene correction. Synthesis of oligonucleotides can be synthesized by any methods known to those skilled in the art.

Antisense RNA (asRNA) technology involves the down-regulation or silencing of gene expression. An “antisense” RNA molecule contains the complement of, and can therefore hybridize with, protein-encoding RNAs of the cell. Antisense oligomers have been shown to bind to messenger RNA at specific sites and inhibit the translation of the RNA into protein, splicing of mRNA or reverse transcription of viral RNA and other processing of mRNA or viral RNA.

RNA silencing is a sequence-specific RNA degradation system that is conserved in a wide range of organisms. RNA silencing is a process by which double-stranded RNA (dsRNA) silences gene expression. Two types of dsRNA involved in RNA silencing include small interfering RNAs (siRNAs) and micro-RNAs (miRNAs) are short double stranded ribonucleic acids that are found in a number of organisms (see Dykxhoorn et al. 2003 Nature Reviews Mol Cell Biol 4:457-466 for a review). SiRNA-like gene silencing mechanisms are functional in virtually all species, including humans. The sequences of many miRNA are known and their positions in the genome or chromosome can be found at http://www.sanger.ac.uk/Software/Rfam/mirna/help/summary.shtml. As of February 2004, this resource lists 176 known human miRNAs and their respective target genes if known.

Although there are some differences in the expression and maturation of siRNAs and miRNAs, the final and active product is in both cases preferably a short, 19-22 nucleotide long, double-stranded RNA molecule (Dykxhoorn et al. 2003 Nature Reviews Mol Cell Biol 4:457-466 and Steinberg 2003 Scientist June 16:22-24). miRNA is synthesized from non-protein coding DNA and is metabolized from transcripts accommodating inverted repeats. The double-stranded RNA formed by foldback is processed by an RNAse III-like enzyme, highly conserved through evolution from yeast to man and higher plants, called Dicer in animals or Dicer-like in plants. These molecules can interact with the 3′-UTRs of transcripts and inhibit translation.

siRNA is cleaved from larger RNA molecules, a process which also involves Dicer. The processing products associate into nucleoprotein complexes, including the RNA-induced silencing complex (RISC), and then bind to mRNA and cause its degradation. Active siRNA is typically formed by two 21-23 nucleotide long ribo-oligonucleotides that form a 19 base pair long duplex with symmetric 2-3 nucleotide long terminal overhangs having 5′phosphate and 3′ hydroxyl groups. As a result of nucleotide sequence homology of siRNA to their cellular target RNA, one strand, usually the antisense strand, binds to the target and renders it inactive and “flagged” for degradation. Thus as is the case for antisenseRNA, the antisense strand of siRNA and miRNA is sufficient for an inhibition of gene expression.

SiRNAs, miRNAs, or as RNAs can be engineered to bind or recognize virtually any RNA target in the cell and thus be used as “knock-down” tools to silence or down-regulate gene expression through RNA inactivation. Methods for the design of siRNAs have been described and are well known in the art (Elbashir et al., Genes Dev., 15:188-200 (2001) and Elbashir, et al., Methods, 26:199-213 (2002)). Means of cellular introduction of antisense RNAs include construction of recombinant vectors which will express antisense RNA once the vector is introduced into the cell, direct injection of the antisense oligonucleotide to cells or tissues, and delivery through anionic and cationic lipid formulations. Chemical modification of the RNA backbone is necessary to achieve a satisfactory half-life in culture for asRNA molecules delivered directly to cells.

The sequences of many siRNA and miRNA molecules are known. In addition, siRNA, miRNA, and asRNA molecules can be designed by methods well known in the art. SiRNA, miRNA, and asRNA molecules with homology sufficient to provide sequence specificity required to uniquely degrade any RNA can be designed using several design programs found on the internet including http://www.ambion.com and http://www.dharmacon.com. The systematic testing of several designed species for optimization of the siRNA, miRNA, and asRNA sequence is routinely performed by those skilled in the art. Considerations when designing short interfering nucleic acid molecules include biophysical, themodynamic, and structural considerations, base preferences at specific positions in the sense strand, and homology. These considerations are well known in the art and provide guidelines for designing siRNA, miRNA, and asRNA molecules for intranasal delivery to the lungs as described in the present application.

b. Aqueous Solutions

The nucleic acid molecules can be prepared in any aqueous carrier, vehicle, or solution so as to provide a composition that is pharmaceutically suitable for in vivo administration. Methods of preparing aqueous solutions are well known to one of ordinary skill in the art. Preferably, the aqueous solutions is water, physiologically acceptable aqueous solutions containing salts and/or buffers, such as phosphate buffered saline (PBS), or any other aqueous solution acceptable for administration to a animal or human. Such solutions are well known to a person skilled in the art and include, but are not limited to, distilled water, de-ionized water, pure or ultrapure water, saline, phosphate-buffered saline (PBS), and solutions containing usual buffers which are compatible with nucleic acids. The compositions may also contain sodium chloride and glucose or mannitol to make the solution isotonic. The composition may contain suitable auxiliary components such as pH, osmolarity and tonicity adjusting agents.

For administration via the upper respiratory tract, the composition is formulated into a solution, e.g., water or isotonic saline, buffered or unbuffered, or as a suspension, at an appropriate concentration for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2 (Remington's Pharmaceutical Sciences 16th edition, Ed. Arthur Osol, page 1445 (1980)). One skilled in the art can readily determine a suitable saline content and pH for an innocuous aqueous solution for nasal and/or upper respiratory administration.

Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

The compositions may contain minor amounts of polymers, surfactants, or other excipients well known to those of the art. In this context, “minor amounts” means no auxiliary agents or substances are present that might affect or mediate uptake of nucleic acid in the cells of the lungs.

II. Adminstration of Compositions to the Respiratory Tract

A. Methods of Administration

Lung-specific delivery of nucleic acid molecule formulations with little to no polymers, surfactants, or excipients as described herein, has diagnostic, prophylactic and therapeutic application for a wide range of lung diseases. Pulmonary administration can typically be completed without the need for medical intervention (self-administration), the pain often associated with injection therapy is avoided, and the amount of enzymatic and pH mediated degradation of the bioactive agent, frequently encountered with oral therapies, is significantly reduced.I

The respiratory tract is the structure involved in the exchange of gases between the atmosphere and the blood stream. The lungs are branching structures ultimately ending with the alveoli where the exchange of gases occurs. The alveolar surface area is the largest in the respiratory system and is where drug absorbtion occurs. The alveoli are covered by a thin epithelium without cilia or a mucus blanket and secrete surfactant phospholipids (J. S. Patton & R. M. Platz. Adv. Drug Del. Rev. 8:179-196 (1992)).

The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchioli which then lead to the ultimate respiratory zone, the alveoli, or deep lung (Gonda, I. “Aerosols for delivery of therapeutic an diagnostic agents to the respiratory tract,” in Critical Reviews in Therapeutic Drug Carrier Systems, 6:273-313 (1990)). The deep lung, or alveoli, are the primary target of inhaled therapeutic aerosols for systemic drug delivery.

Inhaled aerosols have been used for the treatment of local lung disorders including asthma and cystic fibrosis (Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)). Considerable attention has been devoted to the design of therapeutic aerosol inhalers to improve the efficiency of inhalation therapies (Timsina et. al., Int. J Pharm., 101: 1-13 (1995); and Tansey, I. P., Spray Technol. Market, 4: 26-29 (1994)).

Aerosol dosage, formulations and delivery systems may be selected for a particular therapeutic application, as described, for example, in Gonda, I. “Aerosols for delivery of therapeutic and diagnostic agents to the respiratory tract,” in Critical Reviews in Therapeutic Drug Carrier Systems, 6:273-313, 1990; and in Moren, “Aerosol dosage forms and formulations,” in: Aerosols in Medicine, Principles, Diagnosis and Therapy, Moren, et al., Eds. Esevier, Amsterdam, 1985. The term aerosol as used herein refers to any preparation of a fine mist of particles, which can be in solution or a suspension, whether or not it is produced using a propellant. Aerosols can be produced using standard techniques, such as ultrasonication or high pressure treatment.

The formulation may be administered in an aqueous solution that is pharmaceutically acceptable for administration to the respiratory system. Particle sizes greater than 5 μm are deposited in the nasal cavity. Particles that are 2 to 10 μm can be retained in the lungs,and particles of less than 1 μm are exhaled. In preferred embodiments, the compound is administered through inhalation in a form such as liquid particles and/or solid particles. Suitable examples include, but are not limited to, an aerosol, a nebula, a mist, an atomized sample, and liquid drops. Typical apparatus which may be used for administration to humans include metered dose inhalers (MDI), nebulizers, and instillation techniques. The formulation is administered in an amount effective to treat, prevent, or diagnose on one or more symptoms or manifestations of lung disease. It is believed that the nucleic acid molecules can also be administered as dry powders using a dry powder inhaler, where the particles dissolve within the lung secretions.

Various suitable devices and methods of inhalation which can be used to administer particles to a patient's respiratory tract are known in the art. Nebulizers create a fine mist from a solution or suspension, which is inhaled by the patient. The devices described in U.S. Pat. No. 5,709,202 to Lloyd, et al., can be used. An MDI typically includes a pressurized canister having a meter valve, wherein the canister is filled with the solution or suspension and a propellant. The solvent itself may function as the propellant, or the composition may be combined with a propellant, such as freon. The composition is a fine mist when released from the canister due to the release in pressure. The propellant and solvent may wholly or partially evaporate due to the decrease in pressure.

The compositions are preferably delivered into the lung with a pharmacokinetic profile that results in the delivery of an effective dose of the nucleic acid. As generally used herein, an “effective amount” of a nucleic acid of the invention is that amount which is able to treat one or more symptoms of a lung disease, reverse the progression of one or more symptoms of a lung disease, halt the progression of one or more symptoms of a lung disease, prevent the occurence of one or more symptoms of a lung disease, decrease a manifestation of the disease or diagnose one or more symptoms of a lung disease in a subject to whom the compound or therapeutic agent is administered, as compared to a matched subject not receiving the compound or therapeutic agent. The actual effective amounts of drug can vary according to the specific drug or combination thereof being utilized, the particular composition formulated, the mode of administration, and the age, weight, condition of the patient, and severity of the symptoms or condition being treated. Dosages for a particular patient can be determined by one of ordinary skill in the art using conventional considerations, (e.g. by means of an appropriate, conventional pharmacological protocol). In one embodiment, the compositions are delivered at a dose range of 3 to 400 μg per 20 g of body weight, with upper dosing limit of 1 gram per 20 grams body weight. In a preferred embodiment the compositions are delivered at a dose range of 50 to 100 μg per 20 g of body weight. In another preferred embodiment the compositions are delivered at a dose range of 150 nM per kg of body weight.

One or more of these molecules can be administered to an animal (e.g., a human) to modulate expression or activity of one or more target polypeptides. A physician may, for example, prescribe a relatively low dose at first, subsequently increasing the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The efficacy of treatment can be monitored either by measuring the amount of the target gene mRNA (e.g. using real time PCR) or the amount of polypeptide encoded by the target gene mRNA (Western blot analysis).

B. Patients and Diseases to be Treated

The compositions are administered to a patient in need of treatment, prophylaxis or diagnosis. The compositions can be administered to animals or humans. The lung disease may be lung cancer, a respiratory tract or lung infection, a disease of the interstitium, a disorder of gas exchange or blood circulation, a disease of the airways or a disorder of the pleura. As used herein, a “lung cancer⇄ refers to either a primary lung tumor (for example, bronchogenic carcinoma or bronchial carcinoid) or a metastasis from a primary tumor of another organ or tissue (for example, breast, colon, prostate, kidney, thyroid, stomach, cervix, rectum, testis, bone, or melanoma). As used herein, a “respiratory tract or lung infection” refers to any bacterial, viral, fungal, or parasite infection of any part of the respiratory system. As used herein, a “disease of the interstitium” includes any disorder of the interstitium including fibrosis (for example, interstitial pulmonary fibrosis, interstitial pneumonia, interstitial lung disease, Langerhans' cell granulomatosis, sarcoidosis, or idiopathic pulmonary hemosiderosis). As used herein, a “disorder of gas exchange or blood circulation”, refers to any abnormality affecting the distribution and/or exchange of gases to/from the blood and lungs (for example, pulmonary edema, pulmonary embolism, respiratory failure (e.g., due to weak muscles), acute respiratory distress syndrome, or pulmonary hypertension). As used herein, a “disease of the airway” includes any disorder of regular breathing patterns, including disorders of genetic and environmental etiologies (for example, asthma, chronic bronchitis, bronchiolitis, cystic fibrosis, bronchiectasis, emphysema, chronic obstructive pulmonary disease, diffuse panbronchiolitis, or lymphangiomyonatosis). As used herein, a “disorder of the pleura” includes, for example, pleural effusion (e.g., hemothorax (blood into the pleural space), or emphysema (pus into the pleural space), pneumothorax (air, e.g., traumatic, spontaneous, or tension), pleurisy or pleural fibrosis or calcification.

Suitable lung diseases for treatment diagnosis, or prevention of at least one symptom of the lung disease include but are not limited to, lung cancers; lung inflammatory conditions such as asthma, cystic fibrosis, emphysema, bronchitis, and bronchiectasis; interstitial lung disease and interstitial fibrosis; pneumonia caused by bacterial, viral, fungal, parasitic, and mycobacteria infection; occupational lung diseases such as coal, silica, asbestos, and isocyanates; lung disease secondary to collagen vascular diseases such as systemic lupus erythematosis; rheumatoid arthritis; scleroderma; dermatomyositis; mixed connective tissue disorder; vasculitis associated lung disease such as Wegener granulomatosis and Good-pasture's Syndrome; sarcoid; and the syndrome of Acute Lung Injury/Acute Respiratory Distress Syndrome. In a preferred embodiment for systemic delivery, the nucleic acid inhibits expression of heme-oxygenase-1 (HO-1) in the lung and other organs for treatment of congenital hyperbilirubinemia, Crigler-Najjar syndrome, and hyperbilirubinemia of newborns.

### EXAMPLES

### Example 1

### Intranasal Administration of Small Interfering RNA Targeting Heme Oxygenase-1 Following Ischemia-Reperfusion (I-R); Comparison with Viral Vector Mediated Delivery

Intranasal siRNA delivery, without a vector or transfection agent, has lung specificity and heme oxygenase-1 (HO-1) potently regulates lung apoptosis. Systemic HO-1 siRNA may become the basis of modulating severe hyperbilirubinemia of newborns and the severe jaundice of Crigler-Najjar type I patients where there is excessive bilirubin formation and for whom specific therapy does not currently exist.

HO-1 is one of three isoforms of heme oxygenase (HO), the rate-limiting enzyme in the degradation of heme to biliverdin and eventually to bilirubin. HO-1 expression is induced in multiple cell types and organs in response to injury. This induction is postulated to have protective properties; however, the mechanisms remain elusive. HO-2 is primarily constitutive and has been found to be important in the central nervous system. The function of HO-3 is yet unknown. Synthetic heme analogues such as protoporphyrins that competitively inhibit the activity of all HO isoforms are commonly used to study HO-1 function but are limited by the lack of specificity and can have the paradoxical effect of up-regulating HO-1 protein expression. The HO inhibitor tin mesoporphyrin is approved by the Federal Drug Administration for the treatment of hyperbilirubinemia but, again, lacks specificity. The recent emergence of siRNA technology to silence mammalian genes allows for highly specific analysis of gene function and has potential clinical application. Among the limited number of reports on siRNA administration in vivo, all use systemic delivery, transfection chemicals, or viral vectors, and none have been organ-specific to the best of our knowledge (Hasuwa, et al., FEBS Lett., 532:227-230 (2002); McCaffrey, et al., Nature, 418:38-39 (2002); Reich, et al., Mol. Vis., 9:210-216 (2003); Sorensen, et al., J. Mol. Biol., 327:761-766 (2003); and Zender, et al., Proc. Natl. Acad. Sci U.S.A., 100:7797-7802 (2003)).

Previously, the in vivo use of siRNA has been limited. Researchers have found that systemic delivery of caspase 8 siRNA or Fas siRNA into mice protects against liver injury (Zender, et al., Proc. Natl. Acad. Sci. USA 100:7797-7802 (2003) and Song, et al., Nat. Med. 9:347-351 (2003)). Similarly, McCaffrey et al. showed that the administration of hepatitis B virus siRNA effectively inhibited hepatitis B virus replication in cultured cells and mouse liver, indicating that siRNA could be useful in the treatment of viral liver diseases (McCaffrey, et al., Nat. Biotechnol. 21:639-644 (2003). Subretinal siRNA can also be achieved using a chemical transfection agent, and transgenic approaches as well as viral constructs have been used to deliver siRNA in vivo (Reich, et al., Mol. Vis 9:210-216 (2003); Hasuwa, et al., FEBS Lett. 532:227-230 (2002); Rubinson, et al., Nat. Genet. 33:401-406 (2003)). The reports thus far of siRNA in vivo utilize systemic delivery of the siRNA and/or require the use of a transfection chemical or viral vector, which potentially raises concerns for toxicity if used clinically. Furthermore, although systemically delivered siRNA can be detected in multiple organs, including the lung, it is clear that the biologic activity of systemically administered siRNA is not equally effective in all organs. For example, systemic injection of caspase 8 siRNA, although detected in most organs, inhibited Fas-induced liver apoptosis but not lung or kidney apoptosis (Zender, et al., Proc. Natl. Acad. Sci. USA 100:7797-7802 (2003)).

i. Materials and Methods

Primary rat pulmonary artery endothelial cells (PAEC) were maintained in Dulbecco's modified Eagle's medium (Invitrogen) with 10% fetal bovine serum (Hyclone, Logan, Utah) and 0.1% gentamycin (Invitrogen). All data using PAEC were collected before passage 20. PAEC were exposed to anoxia (95% N2.5%CO2) in a sealed modular chamber (Billup-Rothberg, Del Mar, Calif.) with continuous monitoring and automated adjustments to maintain <0.5% O2 (BioSpherix, Redfield, N.Y.) for 24 h. For anoxia-reoxygenation (A-R), the media were changed after 24 h of anoxia, and cells were exposed to normoxia at 37° C. in a humidified atmosphere of 5% CO2 for 1 h. For mouse lung ischemia-reperfusion (I-R), after an intraperitoneal injection of urethane (180 mg/kg of body weight), the mice were intubated via tracheostomy and ventilated with a Harvard ventilator (respiratory rate, 120/min; tidal volume, 0.5 cm). A left hilar clamp was placed for 30 min of unilateral ischemia to the left lung, and the clamp was then released for 2 hours of reperfusion. All of the mouse experiments used male C57B1/6J mice at 6-8 weeks of age weighing 20-25 g as purchased from Jackson Laboratory (Bar Harbor, Me.). The Animal Care and Use Committee at Yale University approved this protocol in accordance with their guidelines.

The design of siRNAs was based on the characterization of siRNA by Elbashir et al. (Elbashir, et al., Genes Dev. 15:188-200 (2001) and Elbashir, et al., Methods 26:199-213 (2002)). siRNAs were synthesized in 2′-deprotected, duplexed, desalted, and purified form by Dharmacon Research, Inc. (Lafayette, Colo.). The sense and antisense strands of rat and mouse HO-1 siRNA were: sequence 1, 5′-AAGGACAUGGCCUUCUGGUAUdTdT-3′ (sense) (SEQ ID NO:1) and 5′-AUACCAGAAGGCCAUGUCCUUdTdT-3′ (antisense) (SEQ ID NO:2); sequence 2, 5′-AAUGAACACUCUGGAGAUGACdTdT-3′ (sense) (SEQ ID NO:3) and 5′-GUCAUCUCCAGAGUGUUCAUUdTdT-3′ (antisense) (SEQ ID NO:4); sequence 3, 5′-AAGACCAGAGUCCCUCACAGAdTdT-3′ (sense) (SEQ ID NO:5) and 5′-UCUGUGAGGGACUCUGGUCUUdTdT-3′ (antisense) (SEQ ID NO:6); sequence 4, 5′-AAGCCACACAGCACUAUGUAAdTdT-3′ (sense) (SEQ ID NO: 7) and 5′-UUACAUAGUGCUGUGUGGCUUdTdT-3′ (antisense) (SEQ ID NO:8); sequence 5, 5′-AAGCCGAGAAUGCUGAGUUCAdTdT-3′ (sense) (SEQ ID NO: 9) and 5′-UGAACUCAGCAUUCUCGGCUUdTdT-3′ (antisense) (SEQ ID NO:10). The sense and antisense strands of human heme oxygenase-1 siRNA were: sequence 6, 5′-GGAGAUUGAGCGCAACAAGdTdT-3′ (sense) (SEQ ID NO:11) and 5′-CUUGUUGCGCUAAAUCUCCdTdT-3′ (antisense) (SEQ ID NO:12); sequence 7, 5′-UGAUAGAAGAGGCCAAGACdTdT-3′ (sense) (SEQ ID NO:13) and 5′-GUCUUGGCCUCUUCUAUCAdTdT-3′ (antisense) (SEQ ID NO:14); sequence 8, 5′-CUGCGUUCCUGCUCAACAUdTdT-3′ (sense) (SEQ ID NO:15)and 5′-AUGUUGAGCAGGAACGCAGdTdT-3′ (antisense) (SEQ ID NO:16). 5′-Biotin-labeled rodent HO-1 siRNA sequence 4 and nonspecific siRNA scrambled duplex, sequence 9, (sense, 5′-GCGCGCUUUGUAGGAUUCGdTdT-3′ (SEQ ID NO:17); antisense, 5′-CGAAUCCUACAAAGCGCGCdTdT-3′ (SEQ ID NO:18)) were also synthesized by Dharmacon Research, Inc. Nonspecific 5′-fluorescein-labeled siRNA, sequence 10, (sense, 5′-UUCUCCGAACGUGUCACGUdTdT-3′ (SEQ ID NO:19); antisense, 5′-ACGUGACACGUUCGGAGAAdTdT-3′ (SEQ ID NO:20)) was synthesized by Qiagen (Germantown, Md.) and used to determine transfection efficiency.

PAEC were seeded into 6-or 12-well plates 1 day prior to transfection using Dulbecco's modified Eagle's tissue culture medium supplemented with 10% fetal bovine serum without antibiotics. At the time of transfection with siRNA, the cells were 50-60% confluent. Oligofectamine reagent (Invitrogen) was used as the transfection agent, and cells were then incubated for 6 h. 30% fetal bovine serum/Dulbecco's modified Eagle' medium was added to reach a final concentration of 10% fetal bovine serum in the wells. Cells were exposed to A-R 24 h after transfection. For in vivo studies, each mouse was anesthetized with methoxyflurane and then given intranasal HO-1 siRNA (2 mg/kg of body weight) or equivalent doses of nonspecific control siRNA duplex or recombinant adenovirus containing rat HO-1 cDNA (Ad-HO-1, a generous gift from Dr. Leo Otterbein, University of Pittsburgh) or the recombinant adenovirus containing the β-galactosidase gene (Ad-X-LacZ, purchased from BD Biosciences Clontech) in a volume of 50 μl.

The human HO-1-expressing replication-deficient retrovirus vector LSN-HHO-1 has been described by Quan, et al., Proc. Natl. Acad. Sci. USA 98:12203-12208 (2001). Exponentially growing PA317 packaging cells were used for transfection and preparation of viral particles. Individual G418-resistant clones were selected, and initial viral titer assays were measured by infecting NIH-3T3 cells as described in Yang, et al., Am. J. Physiol. 277:L127-L133 (1999). PA317/LSN-HHO-1 and the empty viral control cells, PA317/LXSN, were grown until subconfluence was reached. The supernatants were harvested and used to infect PAEC. After selection with G418 a stably transfected cell line of PAEC over-expressing human HO-1, designated LSN/HO-1, and a retroviral vector control cell line, LXSN, were obtained.

For Western blot analysis, protein was extracted from cell or lung tissue lysates, electrotransferred, and immunoblotted with monoclonal HO-1 antibody (Stressgen Biotechnologies, Victoria, Canada) as described (Zhang, et al., Am. J. Physiol. 283:L815-L829 (2002)). Detection was performed with a Phototopehorseradish peroxidase Western detection system (Cell Signaling Technology, Beverly, Mass.). Equivalent sample loading was confirmed by stripping membranes with blot restore membrane rejuvenation solution (Chemicon International, Inc., Temecula, Calif.) and reprobed with anti-β-tubulin antibody.

For apoptosis assays a fluorescence-activated cell sorter (FACS) is used to detect annexin V-fluorescein isothiocyanate labeling (BD Biosciences) according to the manufacturer's instruction. Briefly, after PAEC were washed with cold phosphate-buffered saline and resuspended with binding buffer (10 mM HEPES/NaOH (pH 7.4), 140 mM NaCl, 2.5 mM CaCl2), a solution containing 1×10⁵ cells was transferred to a 5-ml tube, and 5 μl each of annexin V and propidium iodide were added. Binding buffer was then added to each tube and analyzed by FACS (BD Biosciences). The annexin V-fluorescein isothiocyanate signal was detected by FL1 (fluorescein isothiocyanate detector) at 518 nm, and the propidium iodide signal was detected by FL2 (phycoerythrin fluorescence detector) at 620 nm. Mouse lung sections were subjected to terminal deoxynucleotidyltransferase dUTP nick end-la-beling (TUNEL) assay using the in situ cell death detection kit (Roche Diagnostics) as described (Zhang, et al., J. Biol. Chem. 278:1248-1258 (2003)).

For Fas expression PAEC were washed twice in cold phosphate-buffered saline, pelleted, suspended in phosphate-buffered saline containing Fas (1:100 dilution) or control rat IgG (1:100 dilution) antibodies (Santa Cruz Biotechnology, Santa Cruz, Calif.), and incubated on ice for 45 min. The cells were resuspended in phosphate-buffered saline containing anti-rat fluorescein isothiocyanate (1:50 dilution) antibody (Santa Cruz Biotechnology) and fixed in 1% paraformaldehyde prior to FACS analysis. Caspase 3 activity was measured with colorimetric assays using the CaspACE assay system (Promega, Madison, Wis.). PAEC lysates were centrifuged, and the supernatants were incubated with colorimetric substrate, Ac-Asp-Glu-Val-Asp-p-nitroanilide (Ac-DEVD-pNA). The release of p-nitroanilide from Ac-DEVD-pNA was measured at 405 nm using a spectrophotometer.

Data are expressed as mean±S.E. and analyzed by Student's t test. The significant difference was accepted at the p<0.05 level.

ii. Results and Discussion

(a) HO-1 siRNA Inhibits A-R-Induced HO-1 Protein Expression in PAEC

Mouse lungs subjected to I-R injury or endothelial cells exposed to A-R induce HO-1 expression (Zhang, et al., Am. Physiol., 283:L815-L829 (2002)). However, the function of HO-1 during lung I-R injury is unknown. To delineate the role of HO-1 in lung I-R injury, HO-1 induction was knocked down in vitro and in vivo using HO-1 siRNA. Five siRNA sequences (SEQ ID NOS: 1-10) directed against rodent HO-1 were designed according to the methods of Elbashir et al. (Elbashir, et al., Nature 411:494-498 (2001)), and transfected them into PAEC. Sequence 4 (SEQ ID NOS: 7 AND 8) was the most effective in inhibiting A-R-induced HO-1 protein expression. The silencing effect of sequence 4 on HO-1 protein induction during A-R was dose-dependent, whereas incremental doses of nonspecific siRNA had no effect on HO-1 expression. Using 5′-biotin-labeled HO-1 siRNA, efficient entry of the siRNA into PAEC was observed as denoted by the diffuse brown staining in the cytoplasm. Transfection efficiency was quantitated to be greater than 88%, using 5′-fluores (no dash for fluorescein)cein-labeled siRNA detection by FACS. All subsequent in vitro and in vivo studies utilized sequence 4 HO-1 siRNA.

(b) HO-1 siRNA Inhibits I-R-Induced HO-1 Protein Expression in Mouse Lung

The efficacy of the HO-1 siRNA was tested in the mouse lung by intranasal administration. A time course of HO-1 siRNA delivery prior to subjecting the mouse to lung I-R injury shows that HO-1 siRNA has maximal effect in attenuating HO-1 protein induction if given 16 h prior to I-R injury, and persists for at least 72 h. Therefore, intranasal HO-1 siRNA was given for 16 h prior to I-R lung injury in the subsequent mouse experiments. Lung I-R injury causes HO-1 protein induction in other organs such as the kidney, liver, and heart, but intranasal siRNA administration is lung-specific. Intranasal administration of nonspecific siRNA, at the same dose, had no effect on I-R-induced HO-1 expression in the lung or other organs. Specific lung delivery of the siRNA was confirmed by intranasally administering a 5′-biotin-labeled HO-1 siRNA that confers a brown stain to cells that have incorporated the siRNA. Brown-stained cells were detected diffusely in the airway and lung parenchyma as early as 4 h and persisting to 16 h.

(c) HO-1 Overexpression in PAEC and Mouse Lung

To contrast the effects of HO-1 siRNA with HO-1 overexpression, a human HO-1 gene was used in a replication-defective retroviral vector, which has been described previously (Yang, et al., Am. J. Physiol. 277:L127-L133 (1999)), to stably transfect PAEC. Significantly increased HO-1 protein expression in the HO-1 overexpressors (LSN/HO-1) was observed as compared to empty vector transfection (LXSN) in room air. Intranasal administration of rat HO-1 adenovirus (Ad-HO-1) was used to achieve HO-1 overexpression in mouse lungs. HO-1 protein levels at 48 h, 72 h, 5 days, and 1 week were determined after administration of Ad-HO-1 and increased HO-1 protein after 48-72 hours was observed. The empty vector (Ad-X-LacZ) had no effect on HO-1 induction in the mouse lung. To effectively silence the human HO-1 gene in the stably transfected PAEC, three human HO-1 siRNA sequences (1-3) were designed. Transfection experiments revealed that all three siRNA sequences, especially at higher doses, inhibited HO-1 expression in HO-1 overexpressing PAEC if given for 24-72 hours. At 72 hours of siRNA transfection, lower doses (300 nM) of siRNA appeared to be less effective in suppressing HO-1 protein, which is likely because of siRNA degradation. Sequence 1 at 600 nM for 24 h was used in subsequent experiments with human HO-1-overexpressing PAEC (LSN/HO-1).

(d) HO-1 Modulates Apoptosis in PAEC and Mouse Lung During A-R and I-R Injury, Respectively

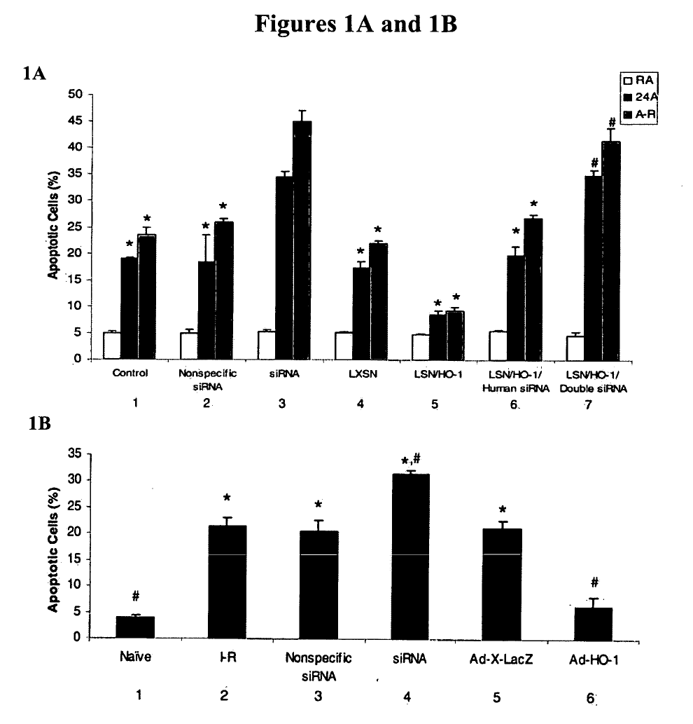

Apoptosis is a pivotal mechanism of I-R-induced organ injury. The biological effect of HO-1 on apoptosis was demonstrated in PAEC during A-R and in mouse lung during I-R. When HO-1 expression was knocked down in PAEC with siRNA directed against rodent HO-1, there was a dramatic increase in A-R-induced apoptosis in PAEC (quantitated by FACS analysis) to 33.8±2.2% during anoxia alone and 41.3±1.4% during A-R compared with wild type controls and cells transfected with nonspecific siRNA (see FIG. 1A, lanes 1-3). Given that the absence of HO-1 induction during A-R was deleterious, HO-1 overexpression might attenuate apoptosis. HO-1-overexpressing cells (LSN/HO-1) subjected to A-R exhibited significantly less apoptosis (8.6±0.2%) during A-R compared with wild type controls and cells that express empty vector (LXSN) (see FIG. 1A, lanes 1, 4, and 5). This effect on apoptosis is specific to HO-1, because the anti-apoptotic effect of HO-1 overexpression was reversed by introducing HO-1 siRNA directed against the human HO-1-overexpressing vector (see FIG. 1A, lane 6). However, given that the HO-1 siRNA was directed against the exogenously transfected human HO-1 gene and considering our previous demonstration that there was endogenous induction of rat HO-1 in PAEC (a rat cell line) during A-R injury (Zhang, et al., Am. J. Physiol. 283:L815-L829 (2002)), the effect of simultaneous inhibition of both rodent and human HO-1 was determined. The presence of both rodent and human siRNA led to more apoptosis than human siRNA alone (41.4±2.4 versus 26.8±0.7%) (see FIG. 1A, lanes 6 and 7, respectively). This was likely because of the fact that even in the presence of human siRNA, which only silenced the transfected human HO-1 gene expression, endogenous HO-1 induction in the PAEC overexpressors continued to have an anti-apoptotic effect. Only when both the exogenous human HO-1 and the endogenous HO-1 induction were silenced did apoptosis reach the levels seen in wild type PAEC treated with rodent HO-1 siRNA (see FIG. 1A, lane 3). Nonspecific siRNA transfection at equivalent doses had no effect on A-R-induced apoptosis. These findings demonstrated the species specificity of the siRNA sequences and highlighted the anti-apoptotic effect of HO-1.

The in vitro findings were correlated with in vivo studies by subjecting mice to lung I-R injury and assessing TUNEL staining. HO-1 siRNA significantly increased the number of TUNEL-positive cells compared with nai\ddot{}ve mice, mice subjected to I-R alone, and mice with nonspecific siRNA subjected to I-R. Mice that overexpressed HO-1 in the lung exhibited significantly less TUNEL staining during lung I-R compared with mice subjected to I-R alone and mice given empty vector (Ad-X-LacZ). The quantitation of TUNEL positive cells paralleled the PAEC results in that HO-1 siRNA in vivo caused significantly increased lung apoptosis, whereas HO-1 overexpression with intranasal administration of HO-1 adenoviral vector decreased apoptosis during lung I-R (see FIG. 1B). Taken together, the data indicate that in both endothelial cells and mouse lung, HO-1 expression has profound anti-apoptotic properties during A-R and I-R injury, respectively.

(e) HO-1 Modulates Apoptosis via Fas and Caspase 3-Dependent Mechanisms in PAEC during A-R Injury

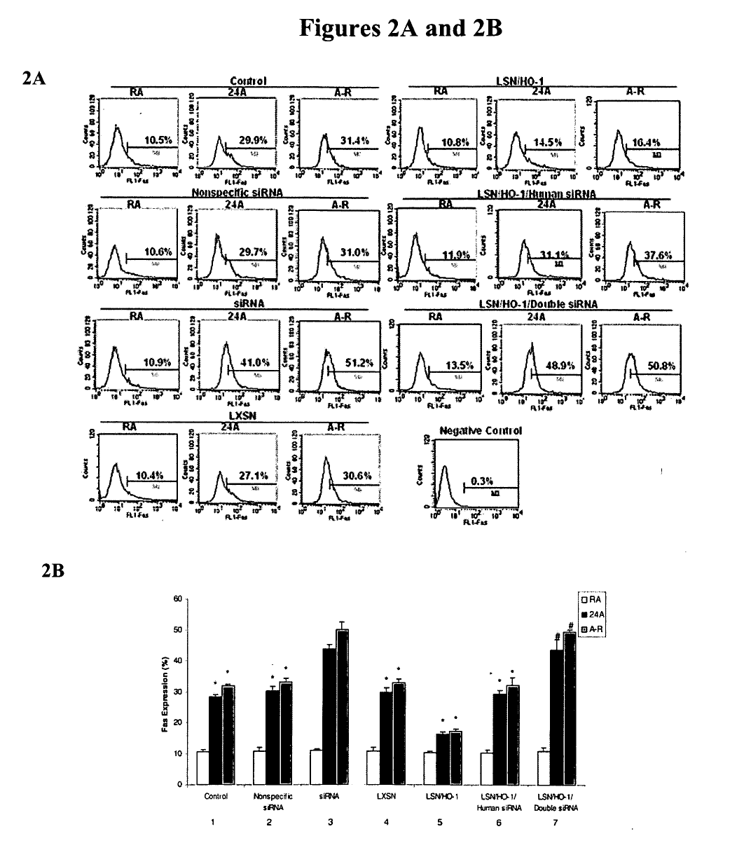

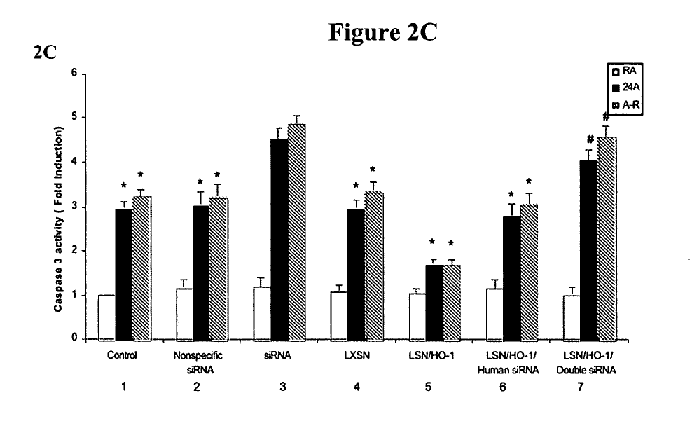

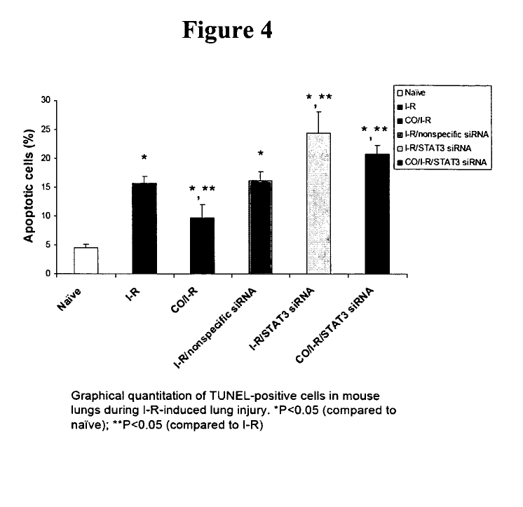

Fas/FasL expression and caspase 3 activity are increased in PAEC during A-R (Zhang, et al., J. Biol. Chem. 278:22061-22070 (2003)). To delineate potential mechanisms of HO-1-modulated apoptosis, the effects of HO-1 siRNA and overexpression on Fas and caspase 3 expression in PAEC during A-R were investigated. FIG. 2A is a representative FACS analysis of Fas expression in PAEC during A-R, which demonstrated that HO-1 siRNA increased A-R-induced Fas expression, whereas stable HO-1 overexpression decreased Fas expression. An antibody to rat IgG was used as a negative control. FIG. 2B is a graphical representation of three independent FACS analyses of Fas expression. The y axis depicts the percentage of cells compared with the total that express Fas during the various conditions. In the presence of HO-1 siRNA (see FIG. 2B, lane 3), there was significantly increased Fas expression (50.2±2.3%) compared with the wild type control cells (31.6±0.9%) and cells transfected with nonspecific siRNA (30.4±1.6%) during A-R. However, in the presence of stable HO-1 overexpression using the human HO-1 gene (LSN/HO-1), there was a dramatic attenuation of A-R-induced Fas expression to 16.9±1.3% compared with cells stably transfected with empty vector (LXSN) (see FIG. 2B, lanes 4 and 5). The effects of HO-1 overexpression on Fas expression were reversed by transfecting the HO-1 overexpressors with siRNA directed against the human HO-1 vector (see FIG. 2B, lane 6). In parallel with the apoptosis studies in PAEC (see FIG. 4A), the silencing of both the human HO-1 overexpression vector and the endogenous rodent HO-1 induction in PAEC resulted in even greater Fas expression in PAEC during A-R (see FIG. 2B, lane 7). In addition to Fas, HO-1 also modulated caspase 3 activity during A-R. HO-1 overexpression significantly attenuated A-R-induced caspase 3 activity in PAEC (1.6±0.1%) (see FIG. 2C, lane 5). On the other hand, HO-1 siRNA, especially if directed against both the exogenous human HO-1 vector and endogenous rodent HO-1, dramatically increased caspase 3 activity in PAEC during A-R (4.6±0.2%) (see FIG. 2C, lane 7).

The highly specific technology of siRNA was used to directly demonstrate that HO-1 has a dramatic effect on apoptosis during lung I-R injury via Fas and caspase 3-dependent pathways. HO-1 overexpression by gene transfer successfully attenuated I-R-induced apoptosis and may potentially have therapeutic value in I-R-induced lung injury for which specific therapies do not currently exist. Lung I-R injury is critical to the pathogenesis of acute lung injury during lung transplantation/surgery, pulmonary embolism, and re-expansion pulmonary edema. The inhibition of apoptosis during I-R injury in other organ systems has been shown to be cytoprotective and to promote organ survival. Therefore, identifying genes that modulate I-R-induced apoptosis would have potential therapeutic relevance to a variety of organ systems. In addition, lung-specific siRNA delivery was demonstrated, without the presence of viral vectors or transfection agents, is biologically effective and may have significant implications for future therapeutic interventions.

The data show that the transfer of exogenous human HO-1 cDNA to endothelial cells or adenovirally-mediated HO-1 over-expression in mouse lung dramatically attenuates A-R-and I-R-induced apoptosis. Conversely, specific knockdown of HO-1 expression using siRNA in vitro and in vivo significantly increased A-R-and I-R-induced apoptosis, respectively. Furthermore, HO-1 overexpression vectors and HO-1 siRNA were used to demonstrate that HO-1 specifically modulates endothelial cell apoptosis by attenuating A-R-induced Fas expression and caspase 3 activity. Thus far, most investigations into the consequences of HO-1 deficiency have utilized synthetic heme analogues such as the protoporphyrins, which are not specific for HO-1 and have the paradoxical effect of inducing HO-1 expression (Jozkowicz, et al., Acta Biochim. Pol. 50:69-79 (2003) and Sardana, et al., Proc. Natl. Acad. Sci. USA 84:2464-2468 (1987)). Reports using HO-1-deficient mice have been limited, likely because of breeding difficulties, and dominant-negative constructs have not been effective. The use of HO-1 siRNA will greatly facilitate the precise identification of the HO-1 gene function.

The data demonstrate that HO-1 siRNA has biologic functions both in vitro as well as in vivo and can be effective in an organ-specific manner. RNA interference mediated by siRNA is a powerful technology that allows the silencing of genes with great specificity and potency. In the present example, intranasal administration of HO-1 siRNA, in the absence of transfection agents, had significant lung-specific effects in modulating apoptosis. The presence of HO-1 siRNA was observed diffusely in the lung airway and alveoli. The use of tissue-specific siRNA with polymerase II rather than polymerase III promoters is also possible, opening up the possibility of targeting specific lung cell types. The ability to apply siRNA in an organ or cell-specific manner is of paramount interest not only for lung diseases but also for a broad range of clinical processes.

### Example 2

### Intranasal Administration of Small Interfering RNA Targeting Heme Oxygenase-1 Following Hyperoxia

i. Materials and Methods

Cell culture and hyperoxia exposures. Murine lung microvascular endothelial cells (MLEC) were isolated from mice lungs with a modification of the methods described by Kuhlencordt et al. 46 and were maintained in 50% DMEM and 50% F12 tissue culture medium supplemented with 20% FBS. Cells were cultured at 37° C. in a humidified atmosphere containing 5% CO2. Hyperoxic conditions were achieved by placing confluent cells in 95% O2/5% CO2 at 37° C. in a tightly sealed modular chamber (Billup-Rothberg, Del Mar, Calif.) for up to 72 h. All experiments were conducted in confluent, quiescent cells that form a monolayer in order to avoid cell density variability between control cells and those exposed to hyperoxia during the course of the experiment.

Animals and hyperoxia exposures. Adult 6 to 8 week old C57BL/6J mice were obtained from Jackson Laboratories (Bar Harbor, Me.). Mice were exposed to 100% O₂ in a Plexiglass exposure chamber and permitted food and water ad libitum. Control mice were exposed to room air under the same conditions. For survival studies, animals were carefully monitored, and the time of death was noted. For the measurement of markers of lung injury, separate groups of mice were removed from the chamber after 72 hrs hyperoxia exposure and killed under anesthesia. Lung specimens were taken for histology, RNA and protein extraction, apoptosis, and immunohistochemistry analyses. All protocols were reviewed and approved by the Animal Care and Use Committee at Yale University.

The design of siRNAs was based on the characterization of siRNA by Elbashir et al. (Elbashir, et al., Genes Dev. 15:188-200 (2001) and Elbashir, et al., Methods 26:199-213 (2002)). siRNAs were synthesized in 2′-Deprotected, Duplexed, Desalted, and Purified form by Dharmacon Research, Inc. (Lafayette, Colo., USA). The sense and anti-sense strands of mouse heme oxygenase-1 (HO-1) siRNA were: Sequence 5: sense: 5′-AAGCCGAGAAUGCUGAGUUCAdTdT-3′ (SEQ ID NO:9), antisense: 5′-UGAACUCAGCAUUCUCGGCUUdTdT-3′ (SEQ ID NO:10); sequence 4: sense: 5′-AAGCCACACAGCACUAUGUAAdTdT-3′ (SEQ ID NO:7), antisense: 5′-UUACAUAGUGCUGUGUGGCUUdTdT-3′ (SEQ ID NO:8). Nonspecific siRNA scrambled duplex, sequence 11, (sense: 5′-GCGCGCUUUGUAGGAUUCGdTdT-3′ (SEQ ID NO:21), antisense: 5′-CGAAUCCUACAAAGCGCGCdTdT-3′ (SEQ ID NO:22)) were also synthesized by Dharmacon Research, Inc.

For transfection of siRNA duplexes in vitro, MLEC were seeded into 6- or 12-well plates one day prior to transfection using 50% DMEM and 50% F12 tissue culture medium supplemented with 20% FBS, without antibiotics. At the time of transfection with HO-1 siRNA, the cells were 50-60% confluent. Oligofectamine Reagent (Invitrogen, Carsbad, Calif., USA) was used as the transfection agent and cells were then incubated for 6 hours. Next, FBS was added to reach a final concentration of 20% FBS in the wells. Cells were exposed to hyperoxia 72 hours after transfection. For in vivo studies, each mouse was anesthetized with methoxyflurane and then given intranasal HO-1 siRNA (2 mg/kg body weight) or equivalent doses of nonspecific control siRNA duplex in a volume of 50 μl.

For RT-PCR analysis, total RNA from lung tissue was extracted by using Trizol reagent (Gibco-BRL), according to the manufacturer's instructions. Primers used for mouse HO-1 were sense: TCCCAGACACCGCTCCTCCAG (SEQ ID NO:23); antisense, GGATTTGGGGCTGCTGGTTTC (SEQ ID NO:24) and for loading control mouse β-actin: sense, GTGGGCCGCTCTAGGCACCAA (SEQ ID NO:25); antisense, CTCTTTGATGTCACGCACGATTTC (SEQ ID NO:26). RT-PCR was performed using RT-PCR Master Mix (USB, Cleveland, Ohio). Conditions for RT-PCR were 1 cycle at 42° C. for 30 min, 1 cycle at 95° C. for 3 min; 30 cycles at 95° C. for 30 s, 60° C. for 1 min, and 68° C. for 1 min 30 s; and 1 cycle at 68° C. for 5 min. Each reaction product (10 μl) was then separated on a 1% agarose gel containing 0.5 μg/ml of ethidium bromide.

For Western Blot Analysis, protein was extracted from cell or lung tissue lysates, electrotransfered, and then immunoblotted with monoclonal HO-1 antibody (Stressgen Biotechnologies, Victoria, BC, Canada). Detection was performed with Phototope-HRP western detection system (Cell Signaling Technology, Beverly, Mass., USA). Equivalent sample loading was confirmed by stripping membranes with Blot Restore Membrane rejuvenation solution (Chemicon International, Inc., Temecula, Calif., USA) and re-probed with anti-μ-tubulin antibody.

ii. Results and Discussion

Hyperoxia induces HO-1 production in vitro and in vivo. Expression levels of HO-1 mRNA and protein were measured by RT-PCR and western blot analysis to determine whether hyperoxia exposure modulates mRNA and protein levels of HO-1. Hyperoxia exposure significantly enhanced the steady-state level of HO-1 mRNA and HO-1 protein in the lungs at 48 and 72 h compared with those in the room air-exposed mice. Hyperoxia-induced lung injury has been extensively investigated and the lung injury is characterized by damage to epithelial and capillary endothelial cells resulting in increased pulmonary capillary permeability, inflammation, cell death, and respiratory failure. Because the lungs possess the largest surface area of endothelial cells in the body and are vulnerable to hyperoxia-induced endothelial apoptosis and lung injury, MLEC were exposed to hyperoxia in vitro. Hyperoxia also significantly increased HO-1 protein expression in vitro from 8 h to 24 h with the peak at 16 h.

HO-1 siRNA inhibits hyperoxia-induced HO-1 protein expression in vitro and in vivo. Mouse lungs subjected to hyperoxia or endothelial cells exposed to hyperoxia induce HO-1 expression. However, the functional significance of HO-1 induction during hyperoxia is not clear. In order to delineate the role of HO-1 in lung hyperoxia-induced injury, HO-1 induction was knocked down in vitro and in vivo using HO-1 siRNA. 2 siRNA sequences directed against mouse HO-1 were designed and transfected into MLEC. Sequence 2 was the most effective in inhibiting hyperoxia-induced HO-1 protein expression, and all subsequent in vitro and in vivo studies utilized sequence 2 HO-1 siRNA. Intranasal administration of HO-1 siRNA into mouse lung successfully inhibited hyperoxia-induced HO-1 expression. Intranasal HO-1 siRNA was given for 3 times at 16 hours prior to hyperoxia, the day exposing to hyperoxia and 24 h later of hyperxia. HO-1 protein level was detected by western blot after 72 h hyperoxia. Intranasal administration significantly inhibited hyperoxia-induced HO-1 protein expression, decreased survival, increased lung injury and apoptosis, whereas nonspecific siRNA administration had no effect on HO-1 expression.

### Example 3

### Intranasal Administration of Small Interfering RNA Targeting Signal Transducer and Activator of Transcription 3 (STAT3) Following Ischemia-Reperfusion (I-R)

Signal Transducers and Activators of Transcription (STATs) are transcription factors that are phosphorylated by JAK kinases in response to cytokine activation of a cell surface receptor tyrosine kinases. Upon activation, the STATs dimerize and are localized to the nucleus where they activate transcription of cytokine-responsive genes. Cytokines that activate STAT3 include growth hormone, interleukin-6 (IL-6) family cytokines, and G-CSF. STAT3 induces progression through the cell cycle, prevents apoptosis and upregulates oncogenes. Activated STATs have been observed in a wide variety of human cancers, including lymphomas and solid tumors. In addition, STAT3 has been shown to play a critical role in hematopoiesis. The importance of STAT3 is underscored by the failure of mice lacking STAT3 to survive embryogenesis. The ability to intranasally administer siRNA targeting STAT3 to the lung was determined. The results demonstrate that the compositions as described herein can be administered intranasally and are not limited to siRNA targeting HO-1.

i. Materials and Methods

For mouse lung ischemia-reperfusion (I-R), all mice, were administered STAT3 siRNA or non-specific siRNA and anesthetized with intraperitoneal injection of urethane (180 mg/kg body weight) and then intubated via tracheostomy and ventilated with Harvard ventilator (respiratory rate=120/min, tidal volume=0.5 cm). A left hilar clamp was placed for 30 minutes of unilateral ischemia to the left lung and then the clamp was released for 2 hours of reperfusion. The Animal Care and Use Committee at Yale University approved this protocol in accordance with the guidelines.

The design of the STAT3 siRNA was based on the characterization of siRNA by Elbashir et al. (Elbashir, et al., Genes Dev. 15:188-200 (2001) and Elbashir, et al., Methods 26:199-213 (2002)). SiRNAs were synthesized in 2′-deprotected, duplexed, desalted, and purified form by Dharmacon Research, Inc. (Lafayette, Colo.). The targeting sequence of mouse STAT3 siRNA was: AACAACCUUCGAAGAAUCAAG (SEQ ID NO:27), and STAT3 siRNA sequence 12 is sense: 5′-CAACCUUCGAAGAAUCAAGUUdTdT-3′ (SEQ ID NO:28), anti-sense: 5′-CUUGAUUCUUCGAAGGUUGUUdtdt-3′ (SEQ ID NO:29). Nonspecific siRNA scrambled duplex is sequence 11 (sense: 5′-GCGCGCUUUGUAGGAUUCGdTdT-3′ (SEQ ID NO:21), antisense: 5′-CGAAUCCUACAAAGCGCGCdTdT-3′ (SEQ ID NO:22)) were also synthesized by Dharmacon Research, Inc. For in vivo studies, each mouse was anesthetized with methoxyflurane and then given intranasal STAT3 siRNA (2 mg/kg body weight) or equivalent doses of nonspecific control siRNA duplex in a volume of 50 μl 16 hours before the performance of lung ischemia-reperfusion.

For Western blot analysis, protein levels of p-STAT3 and STAT3 were analyzed by Western blot assays. Nuclear proteins from lung tissue were extracted, electrotransfered, and then immunoblotted with rabbit phospho-STAT3 antibody or STAT3 antibody (Cell Signaling Technology, Beverly, Mass., USA). Detection was performed with Phototope-HRP western detection system (Cell Signaling Technology, Beverly, Mass., USA). Equivalent sample loading was confirmed by stripping membranes with Blot Restore Membrane rejuvenation solution (Chemicon International, Inc., Temecula, Calif., USA) and re-probed with anti-β-tubulin antibody.

For RT-PCR analysis, total RNA from lung tissue was extracted by using Trizol reagent (Gibco-BRL), according to the manufacturer's instructions. VCAM1, ICAM1, IL-1β, IL-6, IL-10, β-actin, caspase 3, and caspase 8 gene expression were amplified by RT-PCR using RT-PCR Master Mix (USB, Cleveland, Ohio). Conditions for RT-PCR were 1 cycle at 42° C. for 30 min, 1 cycle at 95° C. for 3 min; 30 cycles at 95° C. for 30 s, 60° C. for 1 min, and 68° C. for 1 min 30 s; and 1 cycle at 68° C. for 5 min. Each reaction product (10 μl) was then separated on a 1% agarose gel containing 0.5 μg/ml of ethidium bromide.

For the apopotosis assay, terminal deoxynucleotidyltransferase dUTP nick endlabeling (TUNEL) assay was used with the in situ cell death detection kit (Roche Molecular Biochemicals, Indianapolis, Ind.) to determine the induction of apoptosis in the lungs during I-R-induced lung injury. Caspase 3 activity was measured with colorimetric assays using the CaspACE assay system (Promega, Madison, Wis.), Lung tissues were homogenized in lysis buffer, then centrifuged, and the supernatants were incubated with the colorimetric substrate Ac-Asp-Glu-Val-Asp-p-nitroanilide (Ac-DEVD-pNA). The release of pNA from Ac-DEVD-pNA was measured at 405 nm using a spectrophotometer.

ii. Results and Discussion

The efficacy of the STAT3 siRNA was tested in the mouse lung by intranasal administration. Intranasal STAT3 siRNA was given for 16 h prior to I-R lung injury in the mouse experiments. Lung specificity was not shown directly with the STAT3 siRNA. This is presumed because the methods/composition was nearly identical to that of intranasal HO-1 siRNA.

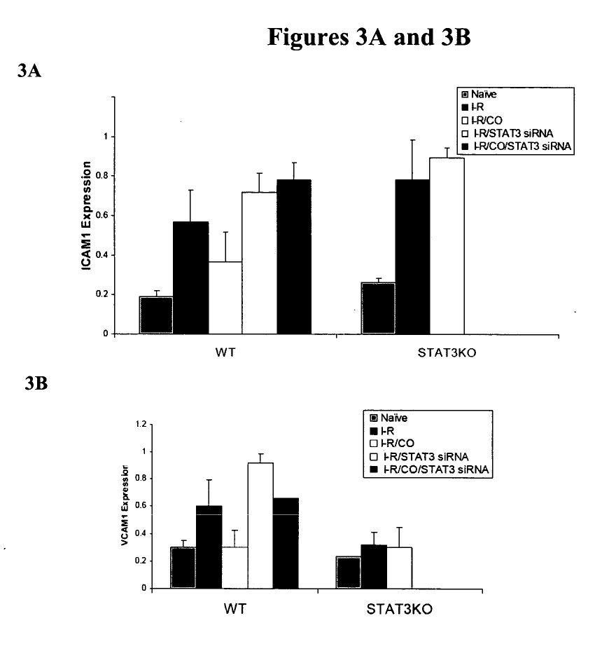

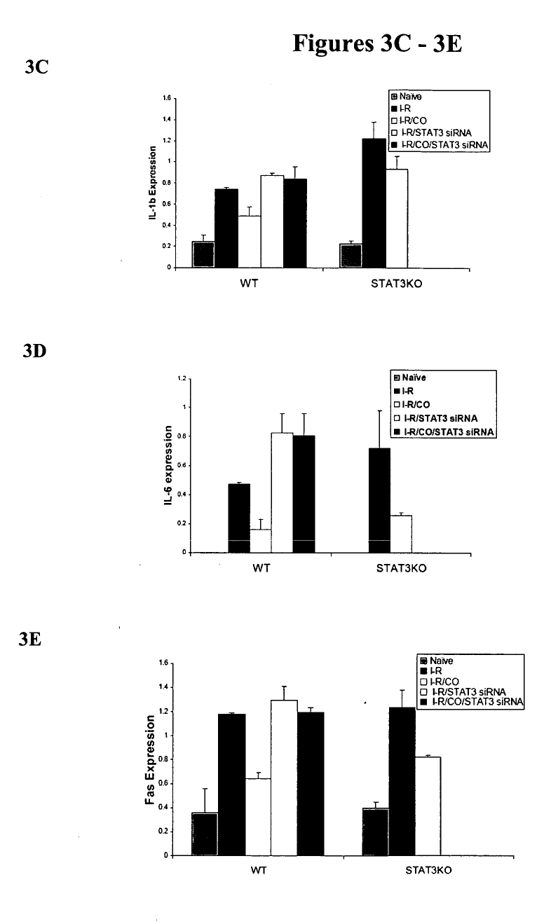

Intranasal administration of STAT3 siRNA increases I-R induced pro-inflammatory gene expression in the lung. Intercellular adhesion molecule 1 (ICAM-1), vascular cell adhesion molecule 1 (VCAM-1), interleukin 1β (IL-1β), interleukin 6 (IL-6), and Fas gene expression were increased in the lung by intranasal administration of STAT3 siRNA after I-R lung injury (see FIGS. 3A, 3B, 3C, 3D, and 3E, respectively). The STAT3 siRNA studies confirm the efficacy of intranasal siRNA in the lung using a gene that is distinct from HO-1 and notably, is an siRNA directed against a transcription factor rather than an enzyme. This illustrates the breadth/range of intranasal siRNA targets.

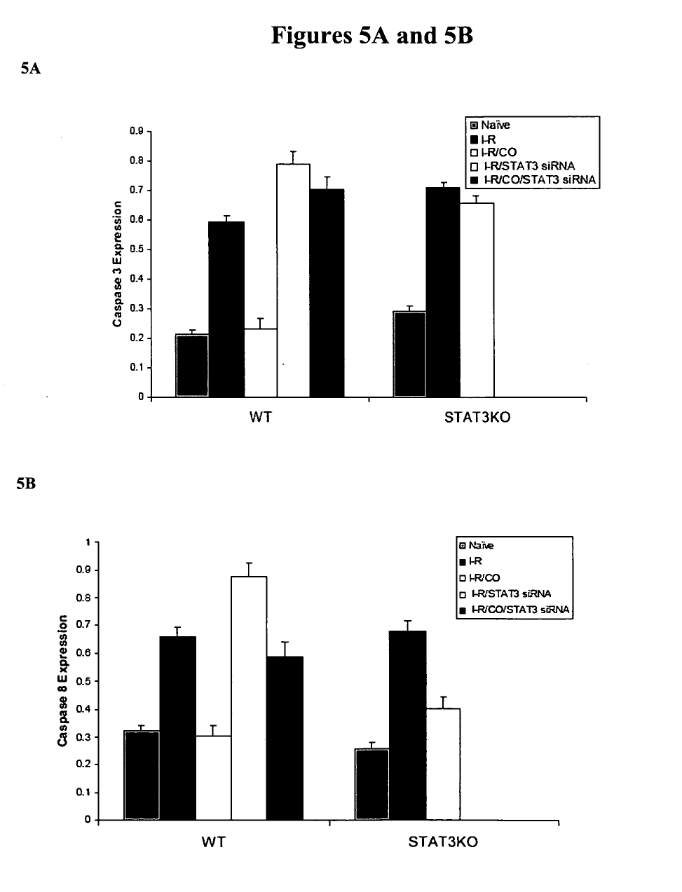

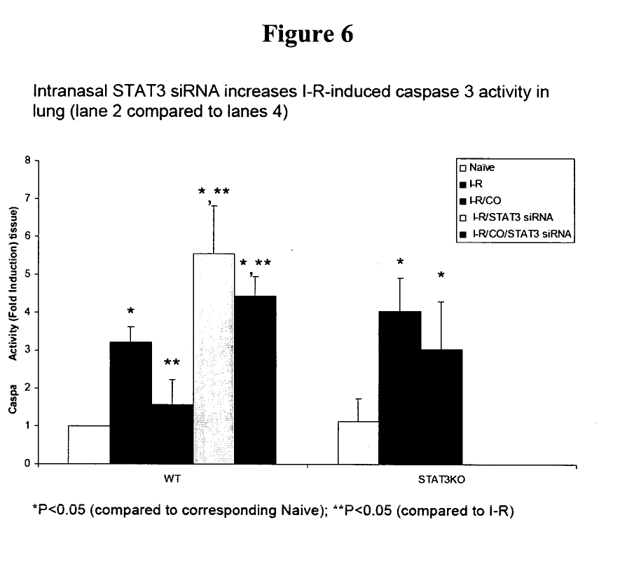

Intranasal administration of STAT3 siRNA also increases I-R induced apopotosis in the lung as determined by the number of TUNEL positive cells (see FIG. 4). To confirm that intranasal administration of STAT3 siRNA increases I-R induced apoptosis in the lung. Quantification of caspase 3 and caspase 8 gene expression during I-R induced in lung injury was performed by using NIH imageJ image processing and analysis software. FIGS. 5A and 5B show that intranasal STAT3 siRNA increases I-R-induced caspases 3 and 8 gene expression in lung. In addition, FIG. 6 shows that intranasal STAT3 siRNA increases I-R-induced caspase 3 activity in the lung.

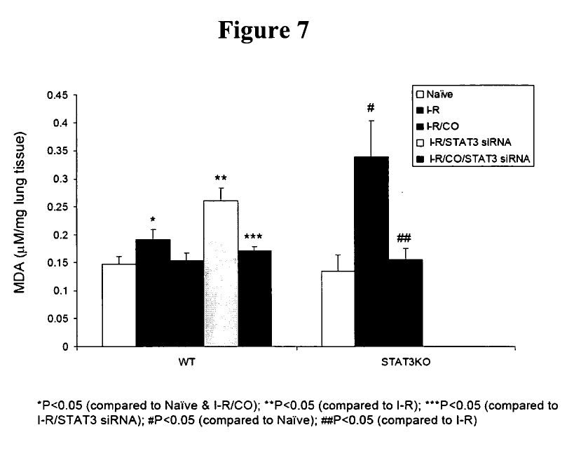

Lipid peroxidation leads to the production of lipid peroxides and their by-products and finally the loss of membrane function and integrity. Malondialdehyde (MDA) are end product derived from peroxidation of polyunsaturated fatty acids and related esters. Measurement of MDA provides a convenient index of lipid peroxidation. MDA was measured in the lung lusates by using lipid peroxidation assay kit (EMD Biosciences, San Diego, Calif.). FIG. 7 shows that intranasal administration of STAT3 siRNA also increases I-R induced lung oxidative stress during I-R injury in the lung as measured by MDA.

### Example 4

### Intranasal Administration of Small Interfering RNA Targeting B cell lymphoma 2 (Bcl-2) Following Hyperoxia Exposure

Bcl-2 belongs to a family of proteins involved in the response to apoptosis. Bcl-2 is anti-apoptotic. The sensitivity of cells to apoptotic stimuli can depend on the balance of pro- and anti-apoptotic proteins. When there is an excess of pro-apoptotic proteins the cells are more sensitive to apoptosis, when there is an excess of anti-apoptotic proteins the cells will tend to be less sensitive. Bcl-2 is an integral membrane protein located in the membranes of the endoplasmic reticulum (ER), nuclear envelope, and in the outer membranes of the mitochondria. The Bcl-2 protein suppresses apoptosis by preventing the activation of the caspases that carry out apoptosis. Mutations in Bcl-2 can give rise to chronic lymphocytic leukemia. Therefore, Bcl-2 is known as an oncogene. Targeting oncogenes for suppression are among the current therapies being developed for treatment of cancers.

Intranasal administration of Bcl-2 siRNA, obtained from Alnylam Pharmaceuticls (Cambridge, Mass.), were performed for mouse and cell hyperoxia exposures as described in example 2. RT-PCR primers for detecting mouse Bcl-2 (NM_{—}009741) is as follows: Detection of long transcript with 3 exons (PCR spanning intron 2).

RT-PCR Product:

(SEQ ID NO:30)

RT-PCR product:
ACCTGAACCGGCATCTGCACACCTGGATCCAGGATAACGGAGGCTGG
GATGCCTTTGTGGAACTATATGGCCCCAGCATGCGACCTCTGTTTGA
TTTCTCCTGGCTG

(SEQ ID NO:31)

Primer: VN-mmBcl2-7:
ACCTGAACCGGCATCTGCACA

(SEQ ID NO:32)

VN-mmBcl2-8:
CAGCCAGGAGAAATCAAACAGAGG

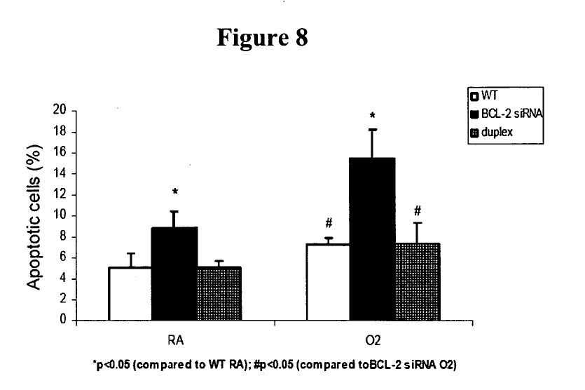

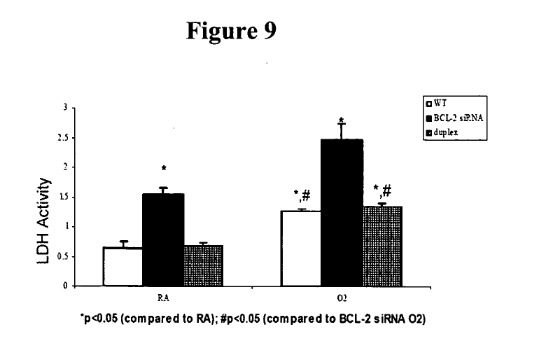

Bcl-2 protein was induced in lung epithelial cells after 24 hour hyperoxia exposure. Intranasal administration of Bcl-2 constructs, B442, B442 mod1, B445, B457, B508, B519 in hyperoxia exposed lung epithelial cells at 100 and 400 nM results in inhibition of Bcl-2 induced expression following hyperoxia exposure in a dose dependent manner. The B442 mod1 construct demonstrated the highest level of inhibition of Bcl-2 induced expression and was therefore used in the subsequent experiments. B442 mod1 construct inhibited Bcl-2 induced expression in lung epithelial cells after 24 hour hyperoxia exposure at 25 nM, 50 nM, and 100 nM doses. To confirm delivery of siRNA constructs to cells, siRNA against GFP labeled with Cy3 was administered intrannasally. Construct delivery was confirmed in lung parenchymal cells (the cell types expressing the fluorescence tag likely includes many cell types including epithelium/in order to specify which cell types would need additional labeling) via fluorescence analysis. Bcl-2 siRNA not only inhibited Bcl-2 hyperoxia induced expression, but also inhibited the number of apoptotic cells induced by hyperoxia in lung epithelial cells (see FIG. 8). In addition, lactate dehydrogenase (LDH) is activated followed hyperoxia exposure (see FIG. 9). Bcl-2 siRNA inhibited hyperoxia induced lactate dehydrogenase activity in lung epithelial cells.

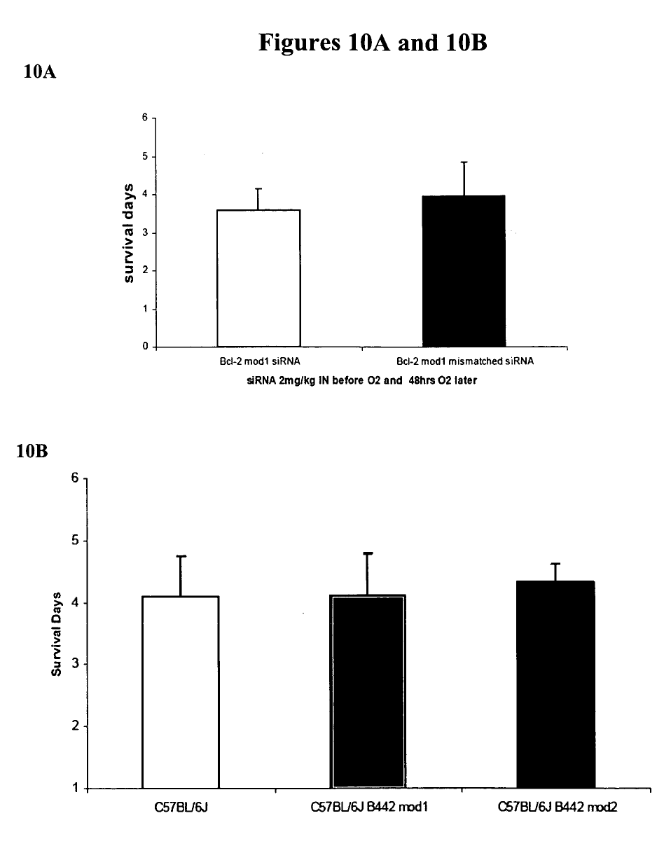

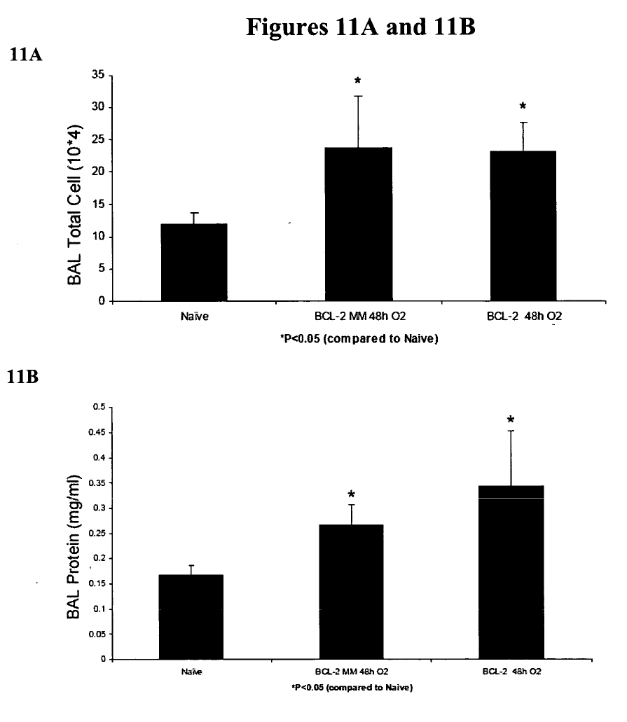

Intranasal administration of Bcl-2 siRNA also decreases hyperoxia-induced Bcl-2 protein and mRNA expression in mouse lung during 24 to 48 hour hyperoxia exposure. Intranasal administration of Bcl-2 siRNA MM (mismatched control) has no effect on hyperoxia-induced Bcl-2 protein expression. FIGS. 10A and 10B show that intranasal administration of Bcl-2 siRNA and its MM (mismatched) control do not change survival in mice during hyperoxia exposure. FIGS. 11A and 11B show that intranasal administration of Bcl-2 siRNA has no effect on hyperoxia-induced lung injury as assessed by lung cell counts or lung protein levels.

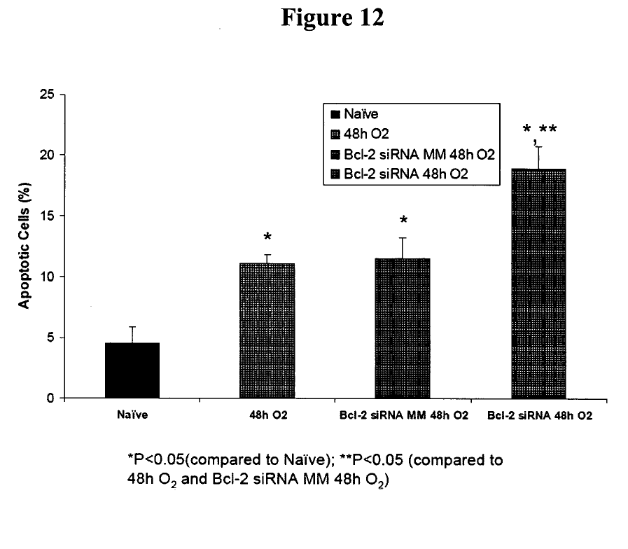

Since intranasal administration of Bcl-2 siRNA in mice had no effect on survival or injury due to hyperoxia exposure theeffect of intranasal administration of Bcl-2 siRNA on apoptosis in mice lung cells was determined. Intranasal administration of Bcl-2 siRNA increased cleaved caspase 3 and cleaved PARP (both markers of apoptosis) during hyperoxia exposure in mouse lung cells. FIG. 12 shows that intranasal administration of Bcl-2 siRNA increases lung apoptosis during hyperoxia exposure as determined by the number of TUNEL positive cells.

### Example 5

### Intranasal Administration of Small Interfering RNA Targeting Vascular Endothelial Growth Factor (VEGF)

VEGF siRNA. The siRNA (sense and antisense strands) were a gift from Alnylam Pharmaceutical and previously published (Filleur et al. Cancer Res. 2003 63:3919-22). The sense and antisense strands for VEGF siRNA sequence 13 are: (sense) 5′-AUGUGAAUGCAGACCAAAGAAdTdT-3′ (SEQ ID NO:33) and (antisense) 5′-UUCUUUGGUCUGCAUUCACAUdTdT-3′ (SEQ ID NO:34). This siRNA matches a 21-nt sequence conserved between the human, rat and mice VEGF mRNA. This siRNA has been successfully used in inhibiting tumor growth by i.p. administration (125 ug/kg/day) (Filleur et al. Cancer Res. 2003 63:3919-22).

Intranasal administration of siRNA. Inducible VEGF transgenic mice were obtained from Jack A Elias lab (Lee et al. Nat Med. 2004(10):1095-103.). Increased levels of human VEGF-165 were noted in BAL even within 24 hours after doxorubicin administration. Each mouse was anesthetized with methoxyflurane and then given intranasal VEGF siRNA (3 nmol/20 g of body weight) in 30 μl PBS daily starting at the same day as doxorubicin turn on. The mice were sacrificed on third day and forth day, respectively.

Transgenic VEGF Quantification. Transgenic VEGF expression levels were quantified by RT-PCR and ELISA. The sequences of primers used in RT-PCR were: sense 5′-CCTCCGCGGCCATGAACTTT-3′ (SEQ ID NO:35) and antisense 5′-TCTTTCCGGATCCGAGATCTGG-3′ (SEQ ID NO:36). These primers are specific to amplify transgenic human VEGF mRNA. ELISA kit for human VEGF was from R&D.

VEGF is one of a number of genes associated with angiogenesis; a process necessary for tumour growth and is a target for treatment of a number of diseases including cancer and other diseases involving angiogenesis.

Intranasal administration of VEGF siRNA, obtained from Alnylam Pharmaceuticals (Cambridge, Mass.) were performed on transgenic mice overexpressing human VEGF in the lung. Intranasal administration of VEGF siRNA decreased human VEGF transgenic overexpression in the mouse lung.

It is understood that the disclosed invention is not limited to the particular methodology, protocols, and reagents described as these may vary. It is also to be understood that the terminology used herein is for the purpose of describing particular embodiments only, and is not intended to limit the scope of the present invention which will be limited only by the appended claims.

Unless defined otherwise, all technical and scientific terms used herein have the same meanings as commonly understood by one of skill in the art to which the disclosed invention belongs. Although any methods and materials similar or equivalent to those described herein can be used in the practice or testing of the present invention, the preferred methods, devices, and materials are as described. Nothing herein is to be construed as an admission that the invention is not entitled to antedate such disclosure by virtue of prior invention.

1. A composition for intranasal delivery wherein the active agent consists essentially of at least one nucleic acid molecule in an effective amount to treat, diagnose, or prevent at least one symptom or manifestation of a disease or disorder when administered intranasally and an aqueous solution.

2. The composition of claim 1 wherein the nucleic acid is less than 30 nucleotides in length.

3. The composition of claim 1 wherein the nucleic acid is in a dose range between 3 to 400 micrograms per 20 grams of body weight.

4. The composition of claim 1 wherein the nucleic acid is DNA or RNA.

5. The composition of claim 4 wherein the RNA is antisense RNA, miRNA, or siRNA.

6. The composition of claim 1 wherein the nucleic acid is single stranded or double stranded.

7. The composition of claim 5 wherein the siRNA inhibits expression of a gene selected from the group consisting of HO-1 STAT3, Bcl-2, and VEGF.

8. The composition of claim 1 wherein the composition is administered in an effective amount to inhibit expression of a target gene in the lung.

9. The composition of claim 1 wherein the nucleic acid is administered in an effective amount to treat, diagnose, or prevent at least one symptom or manifestation of a lung disease.

10. The composition of claim 9 wherein the lung disease is a disease selected from the group consisting of lung cancers; lung inflammatory conditions such as asthma, cystic fibrosis, emphysema, bronchitis, and bronchiectasis; interstitial lung disease and interstitial fibrosis; pneumonia caused by bacterial, viral, fungal, parasitic, and mycobacteria infection; occupational lung diseases such as coal, silica, asbestos, and isocyanates; lung disease secondary to collagen vascular diseases such as systemic lupus erythematosis; rheumatoid arthritis; scleroderma; dermatomyositis; mixed connective tissue disorder; vasculitis associated lung disease such as Wegener granulomatosis and Good-pasture's Syndrome; sarcoid; and the syndrome of Acute Lung Injury/Acute Respiratory Distress Syndrome.

11. A method for treatment, diagnosis, or prevention of at least one symptom or manifestation of a lung disease consisting of administering by intranasal delivery an effective amount of a composition wherein the active ingredient consists essentially of at least one nucleic acid and an aqueous solution.

12. The method of claim 11 wherein the nucleic acid is less than 30 nucleotides in length.

13. The method of claim 11 wherein the composition is administered in a dose range between 3 to 400 micrograms per 20 grams of body weight.

14. The method of claim 11 wherein the nucleic acid is DNA or RNA.

15. The method of claim 14 wherein the RNA is antisense RNA, miRNA, or siRNA.

16. The method of claim 11 wherein the nucleic acid is single stranded or double stranded.

17. The method of claim 11 wherein the siRNA inhibits expression of a gene selected from the group consisting of HO-1 STAT3, Bcl-2, and VEGF.

18. The method of claim 11 wherein the siRNA inhibits expression of HO-1.

19. The method of claim 18 wherein the disease is selected from the group consisting of congenital hyperbilirubinemia, Crigler-Najjar syndrome, and hyperbilirubinemia of newborns.

20. The method of claim 11 wherein the composition is administered in an effective amount to inhibit expression of a target gene in the lung.

21. The method of claim 11 wherein the nucleic acid is administered as a liquid or aerosol.

22. The method of claim 11 wherein the lung disease is a disease selected from the group consisting of lung cancers; lung inflammatory conditions such as asthma, cystic fibrosis, emphysema,bronchitis, and bronchiectasis; interstitial lung disease and interstitial fibrosis; pneumonia caused by bacterial, viral, fungal, parasitic, and mycobacteria infection; occupational lung diseases such as coal, silica, asbestos, and isocyanates; lung disease secondary to collagen vascular diseases such as systemic lupus erythematosis; rheumatoid arthritis; scleroderma; dermatomyositis; mixed connective tissue disorder, vasulitis associated lung disease such as Wegener granulomatosis and Good-pasture's Syndrome; sarcoid; and the syndrome of Acute Lung Injury/Acute Respiratory Distress Syndrome.
